(19) United States
(12) Patent Application Publication
(10) Pub. No.: US 2008/0020123 A1
(43) Pub. Date: Jan. 24, 2008

(54) PLANT MYO-INOSITOL KINASE POLYNUCLEOTIDES AND METHODS OF USE

(75) Inventors: Jinrui Shi, Johnston, IA (US); David Ertl, Waukee, IA (US); Lisa Hagen, West Des Moines, IA (US); Hongyu Wang, Johnston, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 11/839,463

(22) Filed: Aug. 15, 2007

Related U.S. Application Data
(62) Division of application No. 11/132,864, filed on May 19, 2005.
(60) Provisional application No. 60/573,000, filed on May 20, 2004.

(51) Int. Cl. A01H 5/00; A23K 1/00; C12N 15/82; C12N 5/04

(52) U.S. Cl. 426/615; 435/419; 800/278; 800/279; 800/281; 800/298; 800/300; 800/301; 800/302; 800/303

(57) ABSTRACT

Compositions and methods are provided for modulating the level of phytate in plants. More specifically, the invention relates to methods of modulating the level of phytate utilizing nucleic acids comprising myo-inositol kinase (MIK) nucleotide sequences to modulate the expression of MIK(s) in a plant of interest. The compositions and methods of the invention find use in agriculture for improving the nutritional quality of food and feed by reducing the levels of phytate and/or increasing the levels of non-phytate phosphorus in food and feed. The invention also finds use in reducing the environmental impact of animal waste.

### CROSS-REFERENCE TO RELATED APPLICATIONS

This application is a divisional of U.S. application Ser. No. 11/132,864, filed May 19, 2005, which claims the benefit of U.S. Provisional Application No. 60/573,000, filed May 20, 2004, each of which is hereby incorporated in its entirety by reference herein.

### FIELD OF THE INVENTION

The present invention relates to the field of animal nutrition. Specifically, the present invention relates to the identification and use of genes encoding enzymes involved in the metabolism of phytate in plants and the use of these genes and mutants thereof to reduce the levels of phytate, and/or increase the levels of non-phytate phosphorus in food or feed.

### BACKGROUND OF THE INVENTION

The role of phosphorous in animal nutrition is well recognized. Phosphorus is a critical component of the skeleton, nucleic acids, cell membranes and some vitamins. Though phosphorous is essential for the health of animals, not all phosphorous in feed is bioavailable.

Phytates are the major form of phosphorous in seeds. For example, phytate represents about 60-80% of total phosphorous in corn and soybean. When seed-based diets are fed to non-ruminants, the consumed phytic acid forms salts with several important mineral nutrients, such as potassium, calcium, and iron, and also binds proteins in the intestinal tract. These phytate complexes cannot be metabolized by monogastric animals and are excreted, effectively acting as anti-nutritional factors by reducing the bioavailability of dietary phosphorous and minerals. Phytate-bound phosphorous in animal excreta also has a negative environmental impact, contributing to surface and ground water pollution.

There have been two major approaches to reducing the negative nutritional and environmental impacts of phytate in seed. The first involves post-harvest interventions, which increase the cost and processing time of feed. Post-harvest processing technologies remove phytic acid by fermentation or by the addition of compounds, such as phytases.

The second is a genetic approach. One genetic approach involves developing crop germplasm with heritable reductions in seed phytic acid. While some variability for phytic acid was observed, there was no change in non-phytate phosphorous. Further, only 2% of the observed variation in phytic acid was heritable, whereas 98% of the variation was attributed to environmental factors.

Another genetic approach involves selecting low phytate lines from a mutagenized population to produce germplasm. Most mutant lines exhibit a loss of function and are presumably blocked in the phytic acid biosynthetic pathway; therefore, low phytic acid accumulation will likely be a recessive trait. In certain cases, this approach has revealed that homozygosity for substantially reduced phytate can be lethal.

Another genetic approach is transgenic technology, which has been used to increase phytase levels in plants. These transgenic plant tissues or seed have been used as dietary supplements.

The biosynthetic route leading to phytate is complex and not completely understood, and it has been proposed that the production of phytic acid occurs by one of two possible pathways. One possible pathway involves the sequential phosphorylation of Ins(3)P or myo-inositol, leading to the production of phytic acid. Another possible pathway involves hydrolysis of phosphatidylinositol 4,5-bisphosphate by phospholipase C, followed by the phosphorylation of Ins(1,4,5)P₃ by inositol phosphate kinases. This phosphoinositide-mediated pathway is known to occur in mammalian and yeast nuclei, but it has not been shown to operate in the cytosol, where phytic acid is synthesized actively and, in plant seeds, accumulated to high levels. In developing plant seeds, accumulating evidence favors the sequential phosphorylation pathway. Such evidence includes studies of the Lpa2 gene, a gene encoding a maize inositol phosphate kinase which has multiple kinase activities. The Lpa2 gene has been cloned, and the lpa2 mutation has been shown to impair phytic acid synthesis. Mutant lpa2 seeds accumulate myo-inositol and inositol phosphate intermediates.

In plants, as well as in the slime mold Dictyostelium, Ins(3)P is considered to be the start point for a series of phosphorylations which lead to phytic acid. However, it had not been clear whether this Ins(3)P was generated directly from the activity of Ins(3)P synthase or from the activity of myo-inositol kinase. Ins(3)P synthase converts glucose-6-phosphate to Ins(3)P and is the only source of de novo synthesis of Ins(3)P. The dephosphorylation of Ins(3)P, which is catalyzed by inositol monophosphatase, constitutes the sole de novo route to myo-inositol. myo-inositol is essential for cell growth and differentiation and is a precursor for many important metabolites, including phosphoinositides. In plants, myo-inositol can be phosphorylated by myo-inositol kinase (MIK), and this reaction product has been identified as Ins(3)P. When developing seeds were fed tritium-labeled myo-inositol, radioactivity was detected in phytic acid, indicating that phytic acid biosynthesis involves myo-inositol.

Based on the foregoing, there exists the need to improve the nutritional content of plants, particularly corn and soybean, by increasing non-phytate phosphorous and reducing seed phytate. Myo-inositol kinases (MIKs) are involved in the phosphorylation of myo-inositol to make various intermediates in the phytic acid biosynthesis pathway. Accordingly, it is desirable to modulate the expression of MIKs to reduce seed phytate and to increase non-phytate phosphorus.

### SUMMARY OF THE INVENTION

Compositions and methods are provided for modulating the level of phytate in plants. More specifically, the invention relates to methods of modulating the level of phytate utilizing myo-inositol kinase (MIK) nucleic acids to produce transformed plants that exhibit decreased myo-inositol kinase expression. The compositions and methods of the invention find use in agriculture for improving the nutritional quality of food and feed by reducing the levels of phytate and/or increasing the levels of non-phytate phosphorus in food and feed. Thus, the invention finds use in producing food products as well as in reducing the environmental impact of animal waste. Also provided are compositions and methods for producing myo-inositol kinase proteins.

### BRIEF DESCRIPTION OF THE DRAWINGS

FIGS. 1A and 1B show an alignment of the ZmMIK polypeptide (“maize Lpa3”; SEQ ID NO: 2) with a rice protein (“rice”; GenBank Acc. No. AAP03418; SEQ ID NO: 28), an Arabidopsis pfkB family carbohydrate kinase (“Arabidopsis”; GenBank Acc. No. NP_{—}200681; SEQ ID NO: 29), the Sorghum bicolor protein (“sorghum”; an ORF from sorghum BAC genomic sequence, GenBank Acc. No. AF124045; SEQ ID NO: 30), a Brassica oleracea protein (“Brassica”; SEQ ID NO: 31, assembled from three genomic survey sequences, GenBank Acc. Nos. BH473483, BH553276, and BH709390), a sunflower protein C-terminal sequence from EST DH0AG10ZH05RM1 (“sunflower C-term”; GenBank Acc. No. CD857535; SEQ ID NO: 33), a sunflower protein N-terminal sequence from EST QHJ9H03.yg.ab 1 (“sunflower N-term”; GenBank Acc. No. BU036303; SEQ ID NO: 32), and a soybean protein (“soybean”; Pioneer/DuPont EST src3c.pk028.p5:fis; SEQ ID NO: 34), which were identified by a homology search. Letters in bold text indicate a position with identical amino acids among all the sequences, while letters that are shaded indicate conservative changes. The consensus sequence is also shown (SEQ ID NO: 40).

FIG. 2 shows an alignment of the ZmMIK polypeptide (SEQ ID NO: 2) and the pfkB family carbohydrate kinase consensus sequence (pfam00294; SEQ ID NO: 7). The pfam “pfkB family” includes a variety of carbohydrate and pyrimidine kinases. The score of this alignment was −16.4 and the E-value of this alignment was 9.5e⁻⁰⁶. The alignment quality is indicated by the letters and symbols between the two sequences; see, e.g., Bateman et al. (2004) Nucl. Acids Res. 32: D138-D141; Sonnhammer et al. (1997) Proteins 28: 405-420; Bateman et al. (1999) Nucl. Acids Res. 27: 260-262; Sonnhammer et al. (1998) Nucl. Acids. Res. 26: 320-322.

FIG. 3 shows a schematic diagram of the domains of the ZmMIK polypeptide (SEQ ID NO: 2). The consensus sequences for domains A, B, and C are set forth in SEQ ID NOs: 36, 37, and 38, respectively.

FIG. 4 shows an alignment of the ZmMIK polypeptide (“maize Lpa3”; SEQ ID NO: 2) with a rice protein (“rice”; GenBank Acc. No. AAP03418; SEQ ID NO: 28), a sorghum protein (“sorghum”, GenBank Acc. No. AF124045; SEQ ID NO: 30) and an Arabidopsis pfkB family carbohydrate kinase protein (“Arabidopsis”; GenBank Acc. No. NP_{—}200681; SEQ ID NO: 29). The function of these rice, sorghum, and Arabidopsis proteins is not known. Amino acids that are identical in all three proteins are in red text which is also shaded; amino acids that are shared between the rice or Arabidopsis protein and the maize protein are shown in blue text. Conservative changes are shown in black text which is also shaded. The consensus sequence for this alignment (“consensus”) is also shown (SEQ ID NO: 41).

FIG. 5: Diagram of sample constructs. These sample constructs illustrate various configurations that can be used in expression cassettes for use in inhibition of expression, for example, for use in hairpin RNA interference. Sample construct 1 shows a single promoter and fully or partially complementary sequences of “region 1” and “region 2.” Sample construct 2 illustrates a configuration of two sets of fully or partially complementary sequences. In this sample construct, “region 1” is fully or partially complementary to “region 2” and “region 3” is fully or partially complementary to “region 4.” Sample construct 3 illustrates yet another configuration of two sets of fully or partially complementary sequences; here, too, “region 1” is fully or partially complementary to “region 2” and “region 3” is fully or partially complementary to “region 4.”

### DETAILED DESCRIPTION OF THE INVENTION

The invention is drawn to compositions and methods for modulating the level of phytate in plants. Compositions of the invention comprise myo-inositol kinases (“MIKs”) of the invention (i.e., proteins that have myo-inositol kinase activity or “MIK” activity), polynucleotides that encode them, and associated noncoding regions as well as fragments and variants of the exemplary disclosed sequences. For example, the disclosed Lpa3 polypeptides (e.g., SEQ ID NOs: 2 and 6) are MIKs and therefore have myo-inositol kinase activity. The disclosed Lpa3 polynucleotides (e.g., SEQ ID NOs: 1, 3, and 5) encode polypeptides having MIK activity and are therefore “MIK polynucleotides.” In particular, the present invention provides for isolated polynucleotides comprising nucleotide sequences set forth in SEQ ID NOs: 1, 3, or 5 or encoding the amino acid sequences shown in SEQ ID NOs: 2 or 6, and fragments and variants thereof. In addition, the invention provides polynucleotides comprising the complements of these nucleotide sequences. Also provided are polypeptides comprising the sequences set forth in SEQ ID NOs: 28, 29, 30, 31, 32, 33, and 34, polypeptides comprising conserved domains set forth in SEQ ID NOs: 36, 37, and 38, polypeptides comprising the consensus sequences set forth in SEQ ID NOs: 40 and 41, fragments and variants thereof, and nucleotide sequences encoding these polypeptides.

Compositions of the invention also include polynucleotides comprising at least a portion of the promoter sequence set forth in SEQ ID NO: 4 or in nucleotides 1-1379 of SEQ ID NO: 3 as well as polynucleotides comprising other noncoding regions. Also provided is the soybean MIK polynucleotide of SEQ ID NO: 35, which encodes the soybean MIK polypeptide of SEQ ID NO: 34. Thus, the compositions of the invention comprise isolated nucleic acids that encode MIK proteins, fragments and variants thereof, cassettes comprising polynucleotides of the invention, and isolated MIK proteins. The compositions also include nucleic acids comprising nucleotide sequences which are the complement, or antisense, of these MIK nucleotide sequences. The invention further provides plants and microorganisms transformed with these novel nucleic acids as well as methods involving the use of such nucleic acids, proteins, and transformed plants in producing food (including food products) and feed with reduced phytate and/or increased non-phytate phosphorus levels. In some embodiments, the transformed plants of the invention and food and feed produced therefrom have improved nutritional quality due to increased availability (bioavailability) of nutrients including, for example, zinc and iron.

In some embodiments, myo-inositol kinase (“MIK”) activity is reduced or eliminated by transforming a maize plant cell with an expression cassette that expresses a polynucleotide that inhibits the expression of an MIK enzyme such as, for example, an Lpa3 polypeptide. The polynucleotide may inhibit the expression of one or more MIKs directly, by preventing translation of the MIK messenger RNA, or indirectly, by encoding a polypeptide that inhibits the transcription or translation of a maize gene encoding an MIK. Methods for inhibiting or eliminating the expression of a gene in a plant are well known in the art, and any such method may be used in the present invention to inhibit the expression of one or more maize MIKs.

In accordance with the present invention, the expression of an MIK protein is inhibited if the protein level of the MIK is statistically lower than the protein level of the same MIK in a plant that has not been genetically modified or mutagenized to inhibit the expression of that MIK. In particular embodiments of the invention, the protein level of the MIK in a modified plant according to the invention is less than 95%, less than 90%, less than 85%, less than 80%, less than 75%, less than 70%, less than 65%, less than 60%, less than 50%, less than 40%, less than 30%, less than 20%, less than 10%, or less than 5% of the protein level of the same MIK in a plant that is not a mutant or that has not been genetically modified to inhibit the expression of that MIK. The expression level of the MIK may be measured directly, for example, by assaying for the level of MIK expressed in the maize cell or plant, or indirectly, for example, by measuring the activity of the MIK enzyme in the maize cell or plant or by measuring the phytate or Pᵢ level in seeds of the plant. Methods for determining the activity of MIKs are described elsewhere herein; see, e.g., Example 2, and are also described, for example, in Shi et al. (2005) Plant J. published online as doi: 10.1111/j.1365-313X.2005.02412.x. The activity of an MIK protein is “eliminated” according to the invention when it is not detectable by at least one assay method described elsewhere herein.

In other embodiments of the invention, the activity of one or more maize MIKs is reduced or eliminated by transforming a plant cell with an expression cassette comprising a polynucleotide encoding a polypeptide that inhibits the activity of one or more MIKs. The activity of an MIK is inhibited according to the present invention if an MIK activity of the transformed plant or cell is statistically lower than the MIK activity of a plant that has not been genetically modified to inhibit the activity of at least one MIK. In particular embodiments of the invention, the MIK activity of the modified plant according to the invention is less than 95%, less than 90%, less than 85%, less than 80%, less than 75%, less than 70%, less than 65%, less than 60%, less than 50%, less than 40%, less than 30%, less than 20%, less than 10%, or less than 5% of the MIK activity of the same plant that that has not been genetically modified to inhibit the expression of that MIK. MIK activity may be inferred by alterations in phytate content of a transformed plant or plant cell.

In other embodiments, the activity of an MIK may be reduced or eliminated by disrupting the gene encoding the MIK. The invention encompasses mutagenized plants that carry mutations in MIK genes, where the mutations reduce expression of an MIK gene or inhibits the activity of an encoded MIK.

Thus, many methods may be used to reduce or eliminate the activity of an MIK. More than one method may be used to reduce the activity of a single plant MIK. In addition, combinations of methods may be employed to reduce or eliminate the activity of two or more different MIKs. Non-limiting examples of methods of reducing or eliminating the expression of a plant MIK are given below.

In some embodiments of the present invention, a plant cell is transformed with an expression cassette that is capable of producing a polynucleotide that inhibits the expression of MIK. The term “expression” as used herein refers to the biosynthesis of a gene product, including the transcription and/or translation of said gene product. For example, for the purposes of the present invention, an expression cassette capable of expressing a polynucleotide that inhibits the expression of at least one maize MIK is an expression cassette capable of producing an RNA molecule that inhibits the transcription and/or translation of at least one maize MIK.

“Expression” generally refers to the transcription and/or translation of a coding region of a DNA molecule, messenger RNA, or other nucleic acid molecule to produce the encoded protein or polypeptide. In other contexts, “expression” refers to the transcription of RNA from an expression cassette, such as, for example, the transcription of a hairpin construct from an expression cassette for use in hpRNA interference.

“Coding region” refers to the portion of a messenger RNA (or the corresponding portion of another nucleic acid molecule such as a DNA molecule) which encodes a protein or polypeptide. “Noncoding region” refers to all portions of a messenger RNA or other nucleic acid molecule that are not a coding region, including, for example, the promoter region, 5′ untranslated region (“UTR”), and/or 3′ UTR.

Some examples of polynucleotides and methods that inhibit the expression of an MIK are given below. While specific examples are given below, a variety of methods are known in the art by which it is possible to inhibit expression. While the invention is not bound by any particular theory of operation or mechanism of action, the invention provides the exemplary nucleotide and protein sequences disclosed herein and thereby provides a variety of methods by which expression can be inhibited. For example, fragments of noncoding region can be used to make constructs that inhibit expression of an MIK; such fragments can include portions of the promoter region or portions of the 3′ noncoding region (i.e., the 3′ UTR).

In some embodiments of the invention, inhibition of the expression of an MIK may be obtained by sense suppression or cosuppression. For cosuppression, an expression cassette is designed to express an RNA molecule corresponding to all or part of a messenger RNA encoding an MIK in the “sense” orientation. Overexpression of the RNA molecule can result in reduced expression of the native gene. Accordingly, multiple plant lines transformed with the cosuppression expression cassette are screened to identify those that show the greatest inhibition of MIK expression.

The polynucleotide used for cosuppression or other methods to inhibit expression may correspond to all or part of the sequence encoding the MIK, all or part of the 5′ and/or 3′ untranslated region of an MIK transcript, or all or part of both the coding region and the untranslated regions of a transcript encoding MIK. A polynucleotide used for cosuppression or other gene silencing methods may share 99%, 98%, 97%, 96%, 95%, 94%, 93%, 92%, 91%, 90%, 89%, 88%, 87%, 85%, 80%, or less sequence identity with the target sequence. When portions of the polynucleotides are used to disrupt the expression of the target gene, generally, sequences of at least 15, 16, 17, 18, 19, 20, 21, 22, 23, 24, 25, 26, 27, 28, 29, 30, 35, 40, 45, 50, 60, 70, 80, 90, 100, 200, 300, 400, 450, 500, 550, 600, 650, 700, 750, 800, or 900 nucleotides or 1 kb or greater may be used. In some embodiments where the polynucleotide comprises all or part of the coding region for the MIK, the expression cassette is designed to eliminate the start codon of the polynucleotide so that no protein product will be transcribed. In this manner, an expression cassette may cause permanent modification of the coding and/or noncoding region of an endogenous gene.

Thus, in some embodiments, for example, the polynucleotide used for cosuppression or another gene silencing method will comprise a sequence selected from a particular region of the coding and/or noncoding region. That is, the polynucleotide will comprise a sequence or the complement of a sequence selected from the region between nucleotides 1 and 1632 of the sequence set forth in SEQ ID NO: 1, or selected from the region with a first endpoint at nucleotide 1, 90, 150, 250, 400, 550, 700, 850, 1000, 1150, 1300, 1450, or 1632 and a second endpoint at nucleotide 1, 90, 150, 250, 400, 550, 700, 850, 1000, 1150, 1300, 1450, or 1632. As discussed elsewhere herein, fragments and/or variants of the exemplary disclosed sequences may also be used.

In some embodiments, for example, the polynucleotide will comprise a sequence or the complement of a sequence selected from the region between nucleotides 1 and 1379 of the sequence set forth in SEQ ID NO:4, or selected from the region with a first endpoint at nucleotide 1, 150, 250, 400, 550, 700, 850, 1000, 1150, 1300, or 1379 and a second endpoint at nucleotide 1, 150, 250, 400, 550, 700, 850, 1000, 1150, 1300, or 1379. Where a noncoding region is used for cosuppression or other gene silencing method, it may be advantageous to use a noncoding region that comprises CpG islands (see, e.g., Tariq et al. (2004) Trends Genet. 20: 244-251). As discussed elsewhere herein, variants and/or fragments of the exemplary disclosed sequences may also be used.

In some embodiments, for example, the polynucleotide will comprise a sequence or the complement of a sequence selected from the region between nucleotides 1 and 1511 of the sequence set forth in SEQ ID NO:35, or selected from the region with a first endpoint at nucleotide 1, 150, 250, 400, 550, 700, 850, 1000, 1150, 1300, 1450, or 1511 and a second endpoint at nucleotide 1, 150, 250, 400, 550, 700, 850, 1000, 1150, 1300, 1450, or 1511. As discussed elsewhere herein, variants and/or fragments of the exemplary disclosed sequences may also be used. Cosuppression may be used to inhibit the expression of plant genes to produce plants having undetectable protein levels for the proteins encoded by these genes. See, for example, Broin et al. (2002)Plant Cell 14: 1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91: 3490-3496; Jorgensen et al. (1996) Plant Mol. Biol. 31: 957-973; Johansen and Carrington (2001) Plant Physiol. 126: 930-938; Broin et al. (2002)Plant Cell 14: 1417-1432; Stoutjesdijk et al (2002) Plant Physiol. 129: 1723-1731; Yu et al. (2003) Phytochemistry 63: 753-763; and U.S. Pat. Nos. 5,034,323, 5,283,184, and 5,942,657, each of which is herein incorporated by reference. The efficiency of cosuppression may be increased by including a poly-dT region in the expression cassette at a position 3′ to the sense sequence and 5′ of the polyadenylation signal. See, e.g., U.S. Patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

In some embodiments of the invention, inhibition of the expression of the MIK may be obtained by antisense suppression. For antisense suppression, the expression cassette is designed to express an RNA molecule complementary to all or part of a messenger RNA comprising a region encoding the MIK. Overexpression of the antisense RNA molecule can result in reduced expression of the native gene. Accordingly, multiple plant lines transformed with the antisense suppression expression cassette are screened to identify those that show the greatest inhibition of MIK expression.

The polynucleotide for use in antisense suppression may correspond to all or part of the complement of the sequence encoding the MIK, all or part of the complement of the 5′ and/or 3′ untranslated region of the MIK transcript, or all or part of the complement of both the coding sequence and the untranslated regions of a transcript encoding the MIK. In addition, the antisense polynucleotide may be fully complementary (i.e., 100% identical to the complement of the target sequence) or partially complementary (i.e., less than 100% identical to the complement of the target sequence) to the target sequence. That is, an antisense polynucleotide may share 99%, 98%, 97%, 96%, 95%, 94%, 93%, 92%, 91%, 90%, 89%, 88%, 87%, 85%, 80%, or less sequence identity with the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 30, 40, 50, 60, 70, 80, 90, 100, 200, 300, 400, 450, 500, or 550 nucleotides or greater may be used.

Methods for using antisense suppression to inhibit the expression of endogenous genes in plants are described, for example, in Liu et al (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication No. 20020048814, herein incorporated by reference.

In some embodiments of the invention, inhibition of the expression of an MIK may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of MIK expression. Methods for using dsRNA interference to inhibit the expression of endogenous plant genes are described in Waterhouse et al. (1998) Proc. Natl. Acad. Sci. USA 95: 13959-13964, Liu et al. (2002) Plant Physiol. 129: 1732-1743, and WO 99/49029, WO 99/53050, WO 99/61631, and WO 00/49035; each of which is herein incorporated by reference.

In some embodiments of the invention, inhibition of the expression of one or more MIKs may be obtained by hairpin RNA (hpRNA) interference or intron-containing hairpin RNA (hpRNA) interference. These methods are highly efficient at inhibiting the expression of endogenous genes. See, Waterhouse and Helliwell (2003) Nat. Rev. Genet. 4:29-38 and the references cited therein. These methods can make use of either coding region sequences or promoter or regulatory region sequences.

For hpRNA interference, the expression cassette is designed to express an RNA molecule that hybridizes with itself to form a hairpin structure that comprises a single-stranded loop or “spacer” region and a base-paired stem. In some embodiments, the base-paired stem region comprises a sense sequence corresponding to all or part of the endogenous messenger RNA encoding the gene whose expression is to be inhibited, and an antisense sequence that is fully or partially complementary to the sense sequence. The antisense sequence may be located “upstream” of the sense sequence (i.e., the antisense sequence may be closer to the promoter driving expression of the hairpin RNA than the sense sequence). In other embodiments, the base-paired stem region comprises a first portion of a noncoding region such as a promoter and a second portion of the noncoding region that is in inverted orientation and that is fully or partially complementary to the first portion. In some embodiments, the base-paired stem region comprises a first portion and a second portion which are fully or partially complementary to each other but which comprise both coding and noncoding regions.

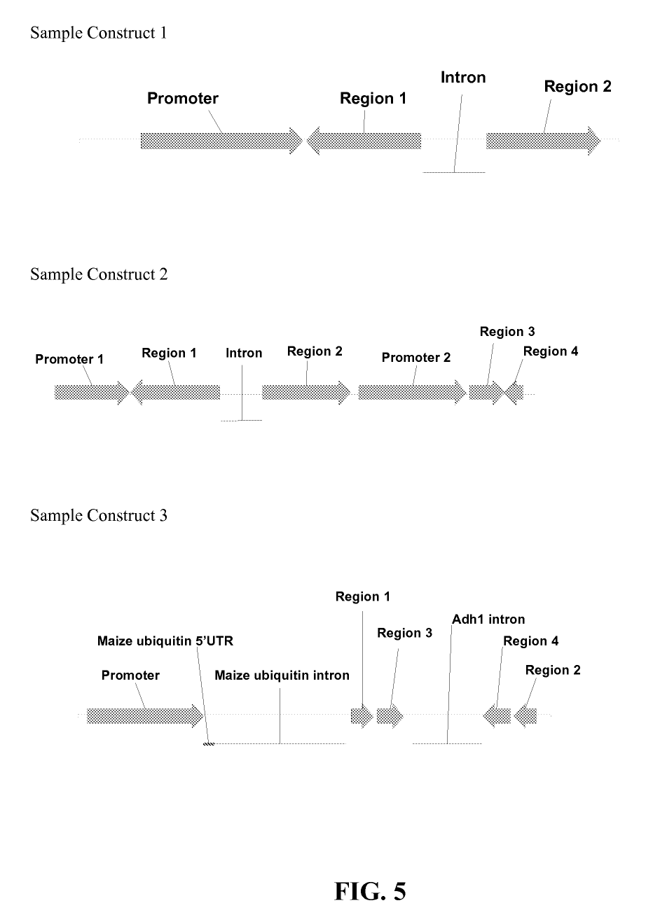

In some embodiments, the expression cassette comprises more than one base-paired “stem” region; that is, the expression cassette comprises sequences from different coding and/or noncoding regions which have the potential to form more than one base-paired “stem” region, for example, as diagrammed in FIG. 5 (construct 2 and construct 3). Where more than one base-paired “stem” region is present in an expression cassette, the “stem” regions may flank one another as diagrammed in FIG. 5 (construct 3) or may be in some other configuration (for example, as diagrammed in FIG. 5 (construct 2)). That is, for example, an expression cassette may comprise more than one combination of promoter and complementary sequences as shown in FIG. 5 (construct 1), and each such combination may be driven by a separate promoter. One of skill will be able to create and test a variety of configurations to determine the optimal construct for use in this or any other method for inhibition of expression.

Thus, the base-paired stem region of the molecule generally determines the specificity of the RNA interference. The sense sequence and the antisense sequence (or first and second portion of the noncoding region) are generally of similar lengths but may differ in length. Thus, either of these sequences may be portions or fragments of at least 10, 19, 20, 30, 50, 70, 90, 100, 120, 140, 160, 180, 200, 220, 240, 260, 280, 300, 320, 340, 360, 380, 400, 500, 600, 700, 800, 900 nucleotides in length, or at least 1, 2, 3, 4, 5, 6, 7, 8, 9, or 10 kb in length. The loop region of the expression cassette may vary in length. Thus, the loop region may be at least 100, 200, 300, 400, 500, 600, 700, 800, 900 nucleotides in length, or at least 1, 2, 3, 4, 5, 6, 7, 8, 9, or 10 kb in length. In some embodiments, the loop region comprises an intron such as, for example, the Adh1 intron.hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97: 4985-4990; Stoutjesdijk et al. (2002) Plant Physiol. 129: 1723-1731; and Waterhouse and Helliwell (2003) Nat. Rev. Genet. 4: 29-38. Methods for using hpRNA interference to inhibit or silence the expression of genes are described, for example, in Chuang and Meyerowitz (2000) Proc. Natl. Acad. Sci. USA 97: 4985-4990; Stoutjesdijk et al. (2002) Plant Physiol. 129: 1723-1731; Waterhouse and Helliwell (2003) Nat. Rev. Genet. 4: 29-38; Pandolfini et al. BMC Biotechnology 3: 7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30: 135-140, herein incorporated by reference. The loop region may vary in length. Thus, the loop region may be at least 100, 200, 300, 400, 500, 600, 700, 800, 900 nucleotides in length, or at least 1, 2, 3, 4, 5, 6, 7, 8, 9, or 10 kb in length.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407: 319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J. 27: 581-590; Wang and Waterhouse (2001) Curr. Opin. Plant Biol. 5: 146-150; Waterhouse and Helliwell (2003) Nat. Rev. Genet. 4: 29-38; Helliwell and Waterhouse (2003) Methods 30: 289-295, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing (Aufsatz et al. (2002) Proc. Nat'l. Acad. Sci. 99 (Suppl. 4): 16499-16506; Mette et al. (2000) EMBO J. 19(19):5194-5201). As the invention is not bound by a particular mechanism or mode of operation, a decrease in expression may also be achieved by other mechanisms.

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for MIK). Methods of using amplicons to inhibit the expression of endogenous plant genes are described, for example, in Angell and Baulcombe (1997) EMBO J. 16: 3675-3684, Angell and Baulcombe (1999) Plant J. 20: 357-362, and U.S. Pat. No. 6,646,805, each of which is herein incorporated by reference.

In some embodiments, the polynucleotide expressed by the expression cassette of the invention is catalytic RNA or has ribozyme activity specific for the messenger RNA of MIK. Thus, the polynucleotide causes the degradation of the endogenous messenger RNA, resulting in reduced expression of the MIK. This method is described, for example, in U.S. Pat. No. 4,987,071, herein incorporated by reference.

In some embodiments of the invention, inhibition of the expression of one or more MIKs may be obtained by RNA interference by expression of a gene encoding a micro RNA (miRNA). miRNAs are regulatory agents consisting of about 22 ribonucleotides. miRNAs are highly efficient at inhibiting the expression of endogenous genes. See, for example Javier et al. (2003) Nature 425: 257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of MIK expression, the 22-nucleotide sequence is selected from an MIK transcript sequence and contains 22 nucleotides of said MIK sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

In one embodiment, the polynucleotide encodes a zinc finger protein that binds to a gene encoding an MIK resulting in reduced expression of the gene. In particular embodiments, the zinc finger protein binds to a regulatory region of an MIK gene. In other embodiments, the zinc finger protein binds to a messenger RNA encoding an MIK and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 20030037355; each of which is herein incorporated by reference.

In some embodiments of the invention, the polynucleotide encodes an antibody that binds to at least one maize MIK and reduces the phytate level of the plant. In another embodiment, the binding of the antibody results in increased turnover of the antibody-MIK complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21: 35-36, incorporated herein by reference. In other embodiments of the invention, the polynucleotide encodes a polypeptide that specifically inhibits the MIK activity of a maize MIK, i.e., a MIK inhibitor.

In some embodiments of the present invention, the activity of an MIK is reduced or eliminated by disrupting the gene encoding the MIK. The gene encoding the MIK may be disrupted by any method known in the art. For example, in one embodiment, the gene is disrupted by transposon tagging. In another embodiment, the gene is disrupted by mutagenizing maize plants using random or targeted mutagenesis, and selecting for plants that have reduced MIK activity.

In one embodiment of the invention, transposon tagging is used to reduce or eliminate the activity of one or more MIKs. Transposon tagging comprises inserting a transposon within an endogenous MIK gene to reduce or eliminate expression of the MIK. “MIK gene” is intended to mean the gene that encodes an MIK protein according to the invention.

In this embodiment, the expression of one or more MIKs is reduced or eliminated by inserting a transposon within a regulatory region or coding region of the gene encoding the MIK. A transposon that is within an exon, intron, 5′ or 3′ untranslated sequence, a promoter, or any other regulatory sequence of an MIK gene may be used to reduce or eliminate the expression and/or activity of the encoded MIK.

Methods for the transposon tagging of specific genes in plants are well known in the art. See, for example, Maes et al. (1999) Trends Plant Sci. 4: 90-96; Dharmapuri and Sonti (1999) FEMS Microbiol. Lett. 179: 53-59; Meissner et al. (2000) Plant J. 22: 265-274; Phogat et al. (2000) J. Biosci. 25: 57-63; Walbot (2000) Curr. Opin. Plant Biol. 2: 103-107; Gai et al. (2000) Nucleic Acids Res. 28: 94-96; Fitzmaurice et al. (1999) Genetics 153: 1919-1928. In addition, the TUSC process for selecting Mu insertions in selected genes has been described in Bensen et al. (1995) Plant Cell 7: 75-84; Mena et al. (1996) Science 274: 1537-1540; and U.S. Pat. No. 5,962,764; each of which is herein incorporated by reference.

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see Ohshima et al. (1998) Virology 243: 472-481; Okubara et al. (1994) Genetics 137: 867-874; and Quesada et al. (2000) Genetics 154: 421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See McCallum et al. (2000) Nat. Biotechnol. 18: 455-457, herein incorporated by reference.

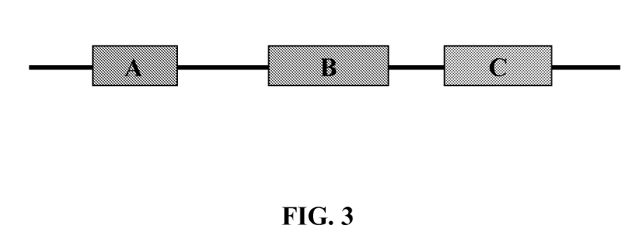

Mutations that impact gene expression or that interfere with the function of the encoded protein are well known in the art. Insertional mutations in gene exons usually result in null-mutants. Mutations in conserved residues are particularly effective in inhibiting the MIK activity of the encoded protein. Conserved residues of plant MIKs suitable for mutagenesis with the goal to eliminate MIK activity are described herein, as shown for example in FIGS. 3 and 6 and in the conserved domains set forth in SEQ ID NOs: 36, 37, 38, 39, 40, and 41. Such mutants can be isolated according to well-known procedures, and mutations in different MIK loci can be stacked by genetic crossing. See, for example, Gruis et al. (2002) Plant Cell 14: 2863-2882.

In another embodiment of this invention, dominant mutants can be used to trigger RNA silencing due to gene inversion and recombination of a duplicated gene locus. See, for example, Kusaba et al. (2003) Plant Cell 15: 1455-1467.

The invention encompasses additional methods for reducing or eliminating the activity of one or more MIKs. Examples of other methods for altering or mutating a genomic nucleotide sequence in a plant are known in the art and include, but are not limited to, the use of chimeric vectors, chimeric mutational vectors, chimeric repair vectors, mixed-duplex oligonucleotides, self-complementary oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham et al. (1999) Proc. Natl. Acad. Sci. USA 96: 8774-8778; each of which is herein incorporated by reference. Other methods of suppressing expression of a gene involve promoter-based silencing. See, for example, Mette et al. (2000) EMBO J. 19: 5194-5201; Sijen et al. (2001) Curr. Biol. 11: 436-440; Jones et al. (2001) Curr. Biol. 11: 747-757.

Where polynucleotides are used to decrease or inhibit MIK activity, it is recognized that modifications of the exemplary sequences disclosed herein may be made as long as the sequences act to decrease or inhibit expression of the corresponding mRNA. Thus, for example, polynucleotides having at least about 70%, 80%, 85%, 86%, 87%, 88%, 89%, 90%, 91%, 92%, 93%, 94%, 95%, 96%, 97%, 98%, 99%, or 100% sequence identity to the exemplary sequences disclosed herein may be used. Furthermore, portions or fragments of the exemplary sequences or portions or fragments of polynucleotides sharing a particular percent sequence identity to the exemplary sequences may be used to disrupt the expression of the target gene. Generally, fragments or sequences of at least 10, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 250, 260, 280, 300, 350, 400, 450, 500, 600, 700, 800, 900, 1000, or more contiguous nucleotides, or greater may be used. It is recognized that in particular embodiments, the complementary sequence of such sequences may be used. For example, hairpin constructs comprise both a sense sequence fragment and a complementary, or antisense, sequence fragment corresponding to the gene of interest. Antisense constructs may share less than 100% sequence identity with the gene of interest, and may comprise portions or fragments of the gene of interest, so long as the object of the embodiment is achieved, i.e., so long as expression of the gene of interest is decreased.

Accordingly, the methods of the invention include methods for modulating the levels of endogenous transcription and/or gene expression by transforming plants with antisense or sense constructs to produce plants with reduced levels of phytate. Generally, such modifications will alter the amino acid sequence of the proteins encoded by the genomic sequence as to reduce or eliminate the activity of a particular endogenous gene, such as MIK, in a plant or part thereof, for example, in a seed.

Furthermore, it is recognized that the methods of the invention may employ a nucleotide construct that is capable of directing, in a transformed plant, the expression of at least one protein, or the transcription of at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or transcription of an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, preferably culture medium represents less than about 30%, 20%, 10%, 5%, or 1% (by dry weight) of chemical precursors or non-protein-of-interest chemicals.

The article “a” and “an” are used herein to refer to one or more than one (i.e., to at least one) of the grammatical object of the article. By way of example, “an element” means one or more element.

Throughout the specification, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

By “modulating” or “modulate” as used herein is intended that the level or amount of a product is increased or decreased in accordance with the goal of the particular embodiment. For example, if a particular embodiment were useful for producing purified MIK enzyme, it would be desirable to increase the amount of MIK protein produced.

Fragments and/or variants of the disclosed polynucleotides and proteins encoded thereby are also encompassed by the present invention. By “fragment” is intended a portion of the polynucleotide or a portion of the nucleotide sequence and hence protein encoded thereby, if any. Fragments of a nucleotide sequence may encode protein fragments that retain the biological activity of the native protein and hence have MIK activity. Alternatively, fragments of a nucleotide sequence that are useful as hybridization probes or in sense or antisense suppression generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of an MIK nucleotide sequence that encodes a biologically active portion of an MIK protein of the invention will encode at least 15, 25, 30, 50, 100, 150, 200, 250, 300, 350, or 360 contiguous amino acids, or up to the total number of amino acids present in a full-length MIK protein of the invention (for example, 379 amino acids for SEQ ID NO: 2). Fragments of an MIK nucleotide sequence that are useful in non-coding embodiments, for example, as PCR primers or for sense or antisense suppression, generally need not encode a biologically active portion of an MIK protein. Thus it will be appreciated that a fragment of an MIK polypeptide of the invention will similarly contain at least 15, 25, 30, 50, 100, 150, 200, 250, 300, 350, or 360 contiguous amino acids, or up to the total number of amino acids present in a full-length MIK protein of the invention (for example, 379 amino acids for SEQ ID NO: 2).

Thus, a fragment of an MIK nucleotide sequence may encode a biologically active portion of an MIK protein, or it may be a fragment that can be used, for example, as a hybridization probe or in sense or antisense suppression using methods disclosed herein and known in the art. A biologically active portion of an MIK protein can be prepared by isolating a portion of one of the MIK polynucleotides of the invention, expressing the encoded portion of the MIK protein (e.g., by recombinant expression in vitro), and assessing the activity of the encoded portion of the MIK protein. Nucleic acid molecules that are fragments of an MIK polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, or 1,600 contiguous nucleotides, or up to the number of nucleotides present in a full-length MIK polynucleotide disclosed herein (for example, 1632 nucleotides for SEQ ID NO: 1).

By “variants” is intended substantially similar sequences. For nucleotide sequences, conservative variants include those sequences that, because of the degeneracy of the genetic code, encode the amino acid sequence of one of the MIK polypeptides of the invention, or a portion thereof. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an MIK protein of the invention, or a portion thereof. Generally, variants of a particular nucleotide sequence of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs described elsewhere herein using default parameters.

Variants of a particular polynucleotide of the invention (i.e., variants of the reference nucleotide sequence) can also be evaluated by comparison of the percent sequence identity between the polypeptide encoded by a variant nucleotide sequence and the polypeptide encoded by the reference nucleotide sequence. Thus, for example, isolated nucleic acids that encode a polypeptide with a given percent sequence identity to the polypeptide of SEQ ID NO: 2, 6, 28, 29, 30, 31, 32, 33, or 34 with a given percent sequence identity to the consensus amino acid sequences of SEQ ID NO: 36, 37, 38, 39, 40, or 41 are disclosed. Percent sequence identity between any two polypeptides can be calculated using sequence alignment programs described elsewhere herein using default parameters. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity. Sequences of the invention may be variants or fragments of an exemplary polynucleotide sequence, or they may be both a variant and a fragment of an exemplary sequence.

“Variants” is intended to mean substantially similar sequences. For polynucleotides, a variant comprises a deletion and/or addition at one or more nucleotides at one or more internal sites within the native polynucleotide and/or a substitution of one or more nucleotides at one or more sites in the native polynucleotide. As used herein, a “native” polypeptide or polynucleotide comprises a naturally occurring amino acid sequence or nucleotide sequence. For polynucleotides, conservative variants include those sequences that, because of the degeneracy of the genetic code, encode the amino acid sequence of one of the MIK polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode an MIK protein of the invention. Generally, variants of a particular polynucleotide of the invention will have at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 2 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity. Sequences of the invention may be variants or fragments of an exemplary polynucleotide sequence, or they may be both a variant and a fragment of an exemplary sequence.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, myo-inositol kinase activity as described herein. Such variants may result from, for example, genetic polymorphism or from human manipulation. Biologically active variants of a native MIK protein of the invention will have at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue. Sequences of the invention may be variants or fragments of an exemplary protein sequence, or they may be both a variant and a fragment of an exemplary sequence.

The proteins of the invention may be altered in various ways including amino acid substitutions, deletions, truncations, and insertions. Methods for such manipulations are generally known in the art. For example, amino acid sequence variants and fragments of the MIK proteins can be prepared by the creation of mutations in the DNA. Methods for mutagenesis and nucleotide sequence alterations are well known in the art. See, for example, Kunkel (1985) Proc. Natl. Acad. Sci. USA 82: 488-492; Kunkel et al. (1987) Methods in Enzymol. 154: 367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Nat'l. Biomed. Res. Found., Washington, D.C.), herein incorporated by reference. Conservative substitutions, such as exchanging one amino acid with another having similar properties, may be made.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired MIK activity. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by the methods used in Examples 1 and 2 and references cited therein as well as by other assays known in the art.

Variant polynucleotides and proteins also encompass sequences and proteins derived from a mutagenic and recombinogenic procedure such as DNA shuffling. With such a procedure, one or more different MIK coding sequences can be manipulated to create a new MIK possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the MIK gene of the invention and other known MIK genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91: 10747-10751; Stemmer (1994) Nature 370: 389-391; Crameri et al. (1997) Nature Biotech. 15: 436-438; Moore et al. (1997) J. Mol. Biol. 272: 336-347; Zhang et al. (1997) Proc. Natl. Acad. Sci. USA 94: 4504-4509; Crameri et al. (1998) Nature 391: 288-291; and U.S. Pat. Nos. 5,605,793 and 5,837,458.

The present invention further provides a method for modulating (i.e., increasing or decreasing) the concentration or composition of the polypeptides of the claimed invention in a plant or part thereof. Modulation can be effected by increasing or decreasing the concentration and/or the composition (i.e., the ratio of the polypeptides of the claimed invention) in a plant.

In some embodiments, the method comprises transforming a plant cell with a cassette comprising a polynucleotide of the invention to obtain a transformed plant cell, growing the transformed plant cell under conditions allowing expression of the polynucleotide in the plant cell in an amount sufficient to modulate concentration and/or composition of the corresponding protein in the plant cell. In some embodiments, the method comprises utilizing the polynucleotides of the invention to create a deletion or inactivation of the native gene. Thus, a deletion may constitute a functional deletion, i.e., the creation of a “null” mutant, or it may constitute removal of part or all of the coding region of the native gene. Methods for creating null mutants are well-known in the art and include, for example, chimeraplasty as discussed elsewhere herein.

In some embodiments, the content and/or composition of polypeptides of the present invention in a plant may be modulated by altering, in vivo or in vitro, the promoter of a non-isolated gene of the present invention to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868. One method of down-regulation of the protein involves using PEST sequences that provide a target for degradation of the protein.

In addition to sense and antisense suppression, catalytic RNA molecules or ribozymes can also be used to inhibit expression of plant genes. The inclusion of ribozyme sequences within antisense RNAs confers RNA-cleaving activity upon them, thereby increasing the activity of the constructs. The design and use of target RNA-specific ribozymes is described in Haseloff et al. (1988) Nature 334: 585-591.

A variety of cross-linking agents, alkylating agents and radical-generating species as pendant groups on polynucleotides of the present invention can be used to bind, label, detect, and/or cleave nucleic acids. For example, Vlassov et al. (1986) Nucl. Acids Res. 14: 4065-4076 describes covalent bonding of a single-stranded DNA fragment with alkylating derivatives of nucleotides complementary to target sequences. Similar work is reported in Knorre et al. (1985) Biochimie 67: 785-789. Others have also showed sequence-specific cleavage of single-stranded DNA mediated by incorporation of a modified nucleotide which was capable of activating cleavage (Iverson and Dervan (1987) J. Am. Chem. Soc. 109: 1241-1243). Meyer et al. ((1989) J. Am. Chem. Soc. 111: 8517-8519) demonstrated covalent crosslinking to a target nucleotide using an alkylating agent complementary to the single-stranded target nucleotide sequence. Lee et al. ((1988) Biochemistry 27: 3197-3203) disclosed a photoactivated crosslinking to single-stranded oligonucleotides mediated by psoralen. Home et al. ((1990) J. Am Chem. Soc. 112: 2435-2437) used crosslinking with triple-helix-forming probes. Webb and Matteucci ((1986) J. Am. Chem. Soc. 108: 2764-2765) and Feteritz et al. ((1991) J. Am. Chem. Soc. 113: 4000) used N4, N4-ethanocytosine as an alkylating agent to crosslink to single-stranded oligonucleotides. In addition, various compounds to bind, detect, label, and/or cleave nucleic acids are known in the art. See, for example, U.S. Pat. Nos. 5,543,507; 5,672,593; 5,484,908; 5,256,648; and 5,681,941. Such embodiments are collectively referred to herein as “chemical destruction.”

In some embodiments, an isolated nucleic acid (e.g., a vector) comprising a promoter sequence is transfected into a plant cell. Subsequently, a plant cell comprising the promoter operably linked to a nucleic acid or polynucleotide comprising a nucleotide sequence of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant-forming conditions for a time sufficient to modulate the concentration and/or composition of polypeptides of the present invention in the plant. Plant forming conditions are well known in the art.

In general, when an endogenous polypeptide is modulated using the methods of the invention, the content of the polypeptide in a plant or part or cell thereof is increased or decreased by at least 5%, 10%, 20%, 30%, 40%, 50%, 60%, 70%, 80%, 90%, or more relative to a native control plant, plant part, or cell lacking the aforementioned cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation.

A plant or plant cell of the invention is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the plant or plant cell of the invention.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire MIK sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated sequences that encode an MIK protein or have Lpa3 promoter activity and which hybridize under stringent conditions to the Lpa3 sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other nucleic acids comprising corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the MIK sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire MIK sequences disclosed herein, or one or more portions thereof, may be used as probes capable of specifically hybridizing to corresponding MIK sequences and messenger RNAs. To achieve specific hybridization under a variety of conditions, such probes include sequences that are unique among MIK sequences and are at least about 10, 12, 15, 16, 17, 18, 19, 20, 21, 22, 23, 24, 25, or 30 nucleotides in length. Such probes may be used to amplify corresponding MIK sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 or 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, “% form” is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). The duration of the wash time will be at least a length of time sufficient to reach equilibrium.

The following terms are used to describe the sequence relationships between two or more nucleic acids or polynucleotides: (a) “reference sequence”, (b) “comparison window”, (c) “sequence identity”, and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, or 100 nucleotides in length, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4: 11-17; the local alignment algorithm of Smith et al. (1981) Adv. Appl. Math. 2: 482; the global alignment algorithm of Needleman and Wunsch (1970) J. Mol. Biol. 48: 443-453; the search-for-local-alignment-method of Pearson and Lipman (1988) Proc. Natl. Acad. Sci. 85: 2444-2448; the algorithm of Karlin and Altschul (1990) Proc. Natl. Acad. Sci. USA 87: 2264, modified as in Karlin and Altschul (1993) Proc. Natl. Acad. Sci. USA 90: 5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73: 237-244 (1988); Higgins et al. (1989) CABIOS 5: 151-153; Corpet et al. (1988) Nucleic Acids Res. 16: 10881-90; Huang et al. (1992) CABIOS 8: 155-65; and Pearson et al. (1994) Meth. Mol. Biol. 24: 307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215: 403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3 and the nwsgapdna.cmp scoring matrix; % identity and % similarity for an amino acid sequence using GAP Weight of 8 and Length Weight of 2; and the BLOSUM62 scoring matrix or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The MIK polynucleotide of the invention can be provided in expression cassettes for expression in the plant of interest. The cassette will include 5′ and 3′ regulatory sequences operably linked to an MIK polynucleotide of the invention. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, “operably linked” is intended to mean that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the MIK polynucleotide to be under the transcriptional regulation of the regulatory regions. The expression cassette may additionally contain selectable marker genes.

Such a cassette is provided with a plurality of restriction sites and/or recombination sites for insertion of the coding sequence to be under the transcriptional control of the regulatory regions. The cassette may additionally contain selectable marker genes. If protein expression is desired, the cassette may be referred to as a protein expression cassette and will include in the 5′-3′ direction of transcription: a transcriptional and translational initiation region (i.e., a promoter), an MIK nucleotide sequence of the invention, and a transcriptional and translational termination region (i.e., termination region) functional in plants.

The regulatory regions (i.e., promoters, transcriptional regulatory regions, and translational termination regions) and/or the MIK polynucleotide of the invention may be native/analogous to the host cell or to each other. Alternatively, the regulatory regions and/or the MIK polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from that from which the polynucleotide was derived, or, if from the same/analogous species, one or both are substantially modified from their original form, or the promoter is not the native promoter for the operably linked polynucleotide.

While it may be optimal to express the sequences using heterologous promoters, the native promoter sequences (e.g., the promoter sequence set forth in SEQ ID NO:4) may be used. Such constructs can change expression levels of MIK in the plant or plant cell. Thus, the phenotype of the plant or plant cell can be altered.

In an expression cassette, the termination region may be native with the transcriptional initiation region, may be native with the operably linked nucleotide sequence of interest, may be native with the plant host, or may be derived from another source (i.e., foreign or heterologous to the promoter, the nucleotide sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17: 7891-7903; and Joshi et al. (1987) Nucleic Acid Res. 15: 9627-9639.

Where appropriate, the polynucleotides may be optimized for increased expression in the transformed plant. That is, the genes can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17: 477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell, and the sequence may be modified to avoid predicted hairpin secondary mRNA structures.

The expression cassettes may additionally contain 5′ leader sequences in the cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86: 6126-6130); potyvirus leaders, for example, TEV leader (Tobacco Etch Virus) (Gallie et al. (1995) Gene 165(2): 233-238), MDMV leader (Maize Dwarf Mosaic Virus) (Virology 154: 9-20), and human immunoglobulin heavy-chain binding protein (BiP) (Macejak et al. (1991) Nature 353: 90-94); untranslated leader from the coat protein mRNA of alfalfa mosaic virus (AMV RNA 4) (Jobling et al. (1987) Nature 325: 622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81: 382-385). See also, Della-Cioppa et al. (1987) Plant Physiol. 84: 965-968. Other methods known to enhance translation can also be utilized, for example, introns, and the like.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85: 610-9 and Fetter et al. (2004) Plant Cell 16: 215-28), cyan florescent protein (CYP) (Bolte et al. (2004) J. Cell Science 117: 943-54 and Kato et al. (2002) Plant Physiol 129: 913-42), and yellow florescent protein (PhiYFP™ from Evrogen; see Bolte et al. (2004) J. Cell Science 117: 943-54).

See generally, Yarranton (1992) Curr. Opin. Biotech. 3: 506-511; Christopherson et al (1992) Proc. Natl. Acad. Sci. USA 89: 6314-6318; Yao et al. (1992) Cell 71: 63-72; Reznikoff (1992) Mol. Microbiol. 6: 2419-2422; Barkley et al. (1980) in The Operon, pp. 177-220; Hu et al. (1987) Cell 48: 555-566; Brown et al. (1987) Cell 49: 603-612; Figge et al. (1988) Cell 52: 713-722; Deuschle et al. (1989) Proc. Natl. Acad. Aci. USA 86: 5400-5404; Fuerst et al. (1989) Proc. Natl. Acad. Sci. USA 86: 2549-2553; Deuschle et al. (1990) Science 248: 480-483; Gossen (1993) Ph.D. Thesis, University of Heidelberg; Reines et al. (1993) Proc. Natl. Acad. Sci. USA 90: 1917-1921; Labow et al. (1990) Mol. Cell. Biol. 10: 3343-3356; Zambretti et al. (1992) Proc. Natl. Acad. Sci. USA 89: 3952-3956; Baim et al. (1991) Proc. Natl. Acad. Sci. USA 88: 5072-5076; Wyborski et al. (1991) Nucleic Acids Res. 19: 4647-4653; Hillenand-Wissman (1989) Topics Mol. Struc. Biol. 10: 143-162; Degenkolb et al. (1991) Antimicrob. Agents Chemother. 35: 1591-1595; Kleinschnidt et al. (1988) Biochemistry 27: 1094-1104; Bonin (1993) Ph.D. Thesis, University of Heidelberg; Gossen et al. (1992)Proc. Natl. Acad. Sci. USA 89: 5547-5551; Oliva et al. (1992) Antimicrob. Agents Chemother. 36: 913-919; Hlavka et al. (1985) Handbook of Experimental Pharmacology, Vol. 78 (Springer-Verlag, Berlin); Gill et al. (1988) Nature 334: 721-724. Such disclosures are herein incorporated by reference.

The above list of selectable marker genes is not meant to be limiting. Any suitable selectable marker gene can be used in the present invention, and one of skill in the art will be able to determine which selectable marker gene is suitable for a particular application.

In preparing the cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313: 810-812); rice actin (McElroy et al. (1990) Plant Cell 2: 163-171); ubiquitin (Christensen et al. (1989) Plant Mol. Biol. 12: 619-632 and Christensen et al. (1992) Plant Mol. Biol. 18: 675-689); pEMU (Last et al. (1991) Theor. Appl. Genet. 81: 581-588); MAS (Velten et al. (1984) EMBO J. 3: 2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Chemical-regulated promoters can be used to modulate the transcription and/or expression of a particular nucleotide sequence in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425 and McNellis et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156), herein incorporated by reference.

Tissue-preferred promoters can be utilized to target enhanced MIK transcription and/or expression within a particular plant tissue. Tissue-preferred promoters include those described in Yamamoto et al. (1997) Plant J. 12(2): 255-265; Kawamata et al. (1997) Plant Cell Physiol. 38(7): 792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol Biol. 23(6): 1129-1138; Matsuoka et al. (1993) Proc Natl. Acad. Sci. USA 90(20): 9586-9590; and Guevara-Garcia et al. (1993) Plant J. 4(3): 495-505. Such promoters can be modified, if necessary, for weak expression.

Leaf-preferred promoters are known in the art. See, for example, Yamamoto et al. (1997) Plant J. 12(2): 255-265; Kwon et al. (1994) Plant Physiol. 105: 357-67; Yamamoto et al. (1994) Plant Cell Physiol. 35(5): 773-778; Gotor et al. (1993) Plant J. 3: 509-18; Orozco et al. (1993) Plant Mol. Biol. 23(6): 1129-1138; and Matsuoka et al. (1993) Proc. Natl. Acad. Sci. USA 90(20): 9586-9590.

Root-preferred promoters are known and can be selected from the many available from the literature or isolated de novo from various compatible species. See, for example, Hire et al. (1992) Plant Mol. Biol. 20(2): 207-218 (soybean root-specific glutamine synthetase gene); Keller and Baumgartner (1991) Plant Cell 3(10): 1051-1061 (root-specific control element in the GRP 1.8 gene of French bean); Sanger et al. (1990) Plant Mol. Biol. 14(3): 433-443 (root-specific promoter of the mannopine synthase (MAS) gene of Agrobacterium tumefaciens); and Miao et al. (1991) Plant Cell 3(1): 11-22 (full-length cDNA clone encoding cytosolic glutamine synthetase (GS), which is expressed in roots and root nodules of soybean). See also Bogusz et al. (1990) Plant Cell 2(7): 633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and rolD root-inducing genes of Agrobacterium rhizogenes (see Plant Science (Limerick) 79(1): 69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2): 343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4): 759-772); and rolB promoter (Capana et al. (1994) Plant Mol. Biol. 25(4): 681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10: 108, herein incorporated by reference. Such seed-preferred promoters include, but are not limited to, Cim1 (cytokinin-induced message); cZ19B1 (maize 19 kDa zein); mi1ps (myo-inositol-1-phosphate synthase); and celA (cellulose synthase) (see WO 00/11177 and U.S. Pat. No. 6,225,529, herein incorporated by reference). Gamma-zein is a preferred endosperm-specific promoter. Globulin (Glb-1) is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed; herein incorporated by reference.

Where low level transcription or expression is desired, weak promoters will be used. Generally, by “weak promoter” is intended a promoter that drives transcription and/or expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of transcription and/or expression. Where a promoter is expressed at unacceptably high levels, portions of the promoter sequence can be deleted or modified to decrease transcription and/or expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

In one embodiment, the polynucleotides of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9: 104-126; Clark et al. (1989) J. Biol. Chem. 264: 17544-17550; Della-Cioppa et al. (1987) Plant Physiol. 84: 965-968; Romer et al. (1993) Biochem. Biophys. Res. Commun. 196: 1414-1421; and Shah et al. (1986) Science 233: 478-481.

Chloroplast targeting sequences are known in the art and include the chloroplast small subunit of ribulose-1,5-bisphosphate carboxylase (Rubisco) (de Castro Silva Filho et al. (1996)Plant Mol. Biol. 30:769-780; Schnell et al. (1991)J. Biol. Chem. 266(5): 3335-3342); 5-(enolpyruvyl)shikimate-3-phosphate synthase (EPSPS) (Archer et al. (1990) J. Bioenerg. Biomemb. 22(6): 789-810); tryptophan synthase (Zhao et al. (1995) J. Biol. Chem. 270(11): 6081-6087); plastocyanin (Lawrence et al. (1997) J. Biol. Chem. 272(33): 20357-20363); chorismate synthase (Schmidt et al. (1993) J. Biol. Chem. 268(36): 27447-27457); and the light harvesting chlorophyll a/b binding protein (LHBP) (Lamppa et al. (1988) J. Biol. Chem. 263: 14996-14999). See also Von Heijne et al. (1991) Plant Mol. Biol. Rep. 9: 104-126; Clark et al. (1989) J. Biol. Chem. 264: 17544-17550; Della-Cioppa et al. (1987) Plant Physiol. 84: 965-968; Romer et al. (1993) Biochem. Biophys. Res. Commun. 196: 1414-1421; and Shah et al. (1986) Science 233: 478-481.

Methods for transformation of chloroplasts are known in the art. See, for example, Svab et al. (1990) Proc. Natl. Acad. Sci. USA 87: 8526-8530; Svab and Maliga (1993) Proc. Natl. Acad. Sci. USA 90: 913-917; Svab and Maliga (1993) EMBO J. 12: 601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91: 7301-7305.

The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See, for example, U.S. Pat. No. 5,380,831, herein incorporated by reference.

In specific embodiments, the MIK sequences of the invention can be provided to a plant using a variety of transient transformation methods. Such transient transformation methods include, but are not limited to, the introduction of the MIK protein or variants and fragments thereof directly into the plant or the introduction of an MIK transcript into the plant. Such methods include, for example, microinjection or particle bombardment. See, for example, Crossway et al. (1986) Mol Gen. Genet. 202: 179-185; Nomura et al. (1986) Plant Sci. 44: 53-58; Hepler et al. (1994) Proc. Natl. Acad. Sci. 91: 2176-2180 and Hush et al. (1994) The Journal of Cell Science 107: 775-784, all of which are herein incorporated by reference. Alternatively, the MIK polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

Thus, transgenic plants having low phytic acid content and high levels of bioavailable phosphorus can be generated by reducing or inhibiting MIK gene expression in a plant. For example, the transgenic plant can contain a transgene comprising an inverted repeat of Lpa3 that suppresses endogenous Lpa3 gene expression. In this manner, transgenic plants having the low phytic acid phenotype of the lpa3 mutant plants can be generated. The transgenic plant can contain an MIK suppressor sequence alone or an MIK suppressor sequence can be “stacked” with one or more polynucleotides of interest, including, for example, one or more polynucleotides that can affect phytic acid levels or that provide another desirable phenotype to the transgenic plant. For example, such a transgene can be “stacked” with similar constructs involving one or more additional genes such as ITPK-5 (inositol 1,3,4-trisphosphate 5/6 kinase; e.g., SEQ ID NO: 45; see also WO 03/027243), IPPK (inositol polyphosphate kinase; e.g., SEQ ID NO: 44; see also WO 02/049324), MRP (e.g., SEQ ID NO: 47; see also copending application entitled “Maize Multidrug Resistance-Associated Protein Polynucleotides and Methods of Use, filed concurrently herewith) and/or a myo-inositol-1 phosphate synthase gene (mi1ps; see U.S. Pat. Nos. 6,197,561 and 6,291,224; e.g., milps-3 (SEQ ID NO: 42)). Transgenes may also be stacked with genes such as phytase (e.g., SEQ ID NO: 48). With such “stacked” transgenes, even greater reduction in phytic acid content of a plant can be achieved, thereby making more phosphorus bioavailable.

Thus, in certain embodiments the nucleic acid sequences of the present invention can be “stacked” with any combination of nucleic acids of interest in order to create plants with a desired phenotype. By “stacked” or “stacking” is intended that a plant of interest contains one or more nucleic acids collectively comprising multiple nucleotide sequences so that the transcription and/or expression of multiple genes are altered in the plant. For example, antisense nucleic acids of the present invention may be stacked with other nucleic acids which comprise a sense or antisense nucleotide sequence of at least one of ITPK-5 (e.g., SEQ ID NO: 45) and/or inositol polyphosphate kinase (IPPK, e.g., SEQ ID NO: 44), IP2K (e.g., SEQ ID NO: 46) or other genes implicated in phytic acid metabolic pathways such as Lpa1 or MRP3 (e.g., SEQ ID NO: 47; see also copending application entitled “Maize Multidrug Resistance-Associated Protein Polynucleotides and Methods of Use, filed concurrently herewith), Lpa2 (see U.S. Pat. Nos. 5,689,054 and 6,111,168); myo-inositol 1-phosphate synthase (milps; e.g., SEQ ID NO: 42), myo-inositol monophosphatase (IMP) (see WO 99/05298 and U.S. application Ser. No. 10/042,465, filed Jan. 9, 2002), and the like. The addition of such nucleic acids could enhance the reduction of phytic acid and InsP intermediates, thereby providing a plant with more bioavailable phosphate and/or reduced phytate. The nucleic acids of the present invention can also be stacked with any other gene or combination of genes to produce plants with a variety of desired trait combinations. For example, in some embodiments, a phytase gene (e.g., SEQ ID NO: 48) is stacked with an lpa1 mutant so that phytase is expressed at high levels in the transgenic plant. Phytase genes are known in the art. See, for example, Maugenest et al. (1999) Plant Mol. Biol. 39: 503-514; Maugenest et al. (1997) Biochem. J. 322: 511-517; WO 200183763; WO200200890.

An MIK polynucleotide also can be stacked with any other polynucleotide(s) to produce plants having a variety of desired trait combinations including, for example, traits desirable for animal feed such as high oil genes (see, e.g., U.S. Pat. No. 6,232,529, which is incorporated herein by reference); balanced amino acids (e.g., hordothionins; see U.S. Pat. Nos. 5,990,389; 5,885,801; 5,885,802; and 5,703,409, each of which is incorporated herein by reference); barley high lysine (Williamson et al. (1987) Eur. J. Biochem. 165: 99-106 and WO 98/20122); high methionine proteins (Pedersen et al. (1986) J. Biol. Chem. 261: 6279; Kirihara et al. (1988) Gene 71: 359; and Musumura et al. (1989) Plant Mol. Biol. 12: 123); increased digestibility (e.g., modified storage proteins) and thioredoxins (U.S. Ser. No. 10/005,429, filed Dec. 3, 2001).

An MIK polynucleotide also can be stacked with one or more polynucleotides encoding a desirable trait such as a polynucleotide that confers, for example, insect, disease or herbicide resistance (e.g., Bacillus thuringiensis toxic proteins; U.S. Pat. Nos. 5,366,892; 5,747,450; 5,737,514; 5,723,756; 5,593,881; Geiser et al. (1986) Gene 48: 109); lectins (Van Damme et al. (1994) Plant Mol. Biol. 24: 825); fumonisin detoxification genes (U.S. Pat. No. 5,792,931); avirulence and disease resistance genes (Jones et al. (1994) Science 266: 789; Martin et al. (1993) Science 262: 1432; Mindrinos et al. (1994) Cell 78: 1089); acetolactate synthase mutants that lead to herbicide resistance such as the S4 and/or Hra mutations; inhibitors of glutamine synthase such as phosphinothricin or basta (e.g., the bar gene); and glyphosate (e.g., the EPSPS gene and the GAT gene; see, for example, U.S. Publication No. 20040082770 and WO 03/092360) or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Additional polynucleotides that can be stacked with a MIK polynucleotide include, for example, those encoding traits desirable for processing or process products such as modified oils (e.g., fatty acid desaturase genes (U.S. Pat. No. 5,952,544; WO 94/11516); modified starches (e.g., ADPG pyrophosphorylases, starch synthases, starch branching enzymes, and starch debranching enzymes); and polymers or bioplastics (e.g., U.S. Pat. No. 5,602,321). An MIK polynucleotide of the invention also can be stacked with one or more polynucleotides that provide desirable agronomic traits such as male sterility (e.g., U.S. Pat. No. 5,583,210), stalk strength, flowering time, or transformation technology traits such as cell cycle regulation or gene targeting (e.g., WO 99/61619; WO 00/17364; WO 99/25821). Other desirable traits that are known in the art include high oil content; increased digestibility; balanced amino acid content; and high energy content. Such traits may refer to properties of both seed and non-seed plant tissues, or to food or feed prepared from plants or seeds having such traits; such food or feed will have improved quality.

These stacked combinations can be created by any method including but not limited to cross breeding plants by any conventional or TopCross methodology, or genetic transformation. In this regard, it is understood that transformed plants of the invention include a plant that contains a sequence of the invention that was introduced into that plant via breeding of a transformed ancestor plant. If the traits are stacked by genetically transforming the plants, the nucleic acids of interest can be combined at any time and in any order. Similarly, where a method requires more than one step to be performed, it is understood that steps may be performed in any order that accomplishes the desired end result. For example, a transgenic plant comprising one or more desired traits can be used as the target to introduce further traits by subsequent transformation. The traits can be introduced simultaneously in a co-transformation protocol with the polynucleotides of interest provided by any combination of cassettes suitable for transformation. For example, if two sequences will be introduced, the two sequences can be contained in separate cassettes (trans) or contained on the same transformation cassette (cis). Transcription and/or expression of the sequences can be driven by the same promoter or by different promoters. In certain cases, it may be desirable to introduce a cassette that will suppress the expression of the polynucleotide of interest. This may be combined with any combination of other cassettes to generate the desired combination of traits in the plant. Alternatively, traits may be stacked by transforming different plants to obtain those traits; the transformed plants may then be crossed together and progeny may be selected which contains all of the desired traits.

Stacking may also be performed with fragments of a particular gene or nucleic acid. In such embodiments, a plants is transformed with at least one fragment and the resulting transformed plant is crossed with another transformed plant; progeny of this cross may then be selected which contain the fragment in addition to other transgenes, including, for example, other fragments. These fragments may then be recombined or otherwise reassembled within the progeny plant, for example, using site-specific recombination systems known in the art. Such stacking techniques could be used to provide any property associated with fragments, including, for example, hairpin RNA (hpRNA) interference or intron-containing hairpin RNA (ihpRNA) interference.

It is understood that in some embodiments the nucleic acids to be stacked with MIK can also be designed to reduce or eliminate the expression of a particular protein, as described in detail herein for MIK. Thus, the methods described herein with regard to the reduction or elimination of expression of MIK are equally applicable to other nucleic acids and nucleotide sequences of interest, such as, for example, IPPK, ITPK-5, and mi1ps, examples of which are known in the art and which are expected to exist in most varieties of plants. Accordingly, the descriptions herein of MIK fragments, variants, and other nucleic acids and nucleotide sequences apply equally to other nucleic acids and nucleotide sequences of interest such as milps, IPPK, or ITPK-5. For example, an antisense construct could be designed for milps comprising a nucleotide sequence that shared 90% sequence identity to the complement of SEQ ID NO: 42 or was a 50-nucleotide fragment of the complement of SEQ ID NO: 42.

Transformation protocols as well as protocols for introducing polypeptides or polynucleotides into plants may vary depending on the type of plant or plant cell, i.e., monocot or dicot, targeted for transformation. Suitable methods of introducing polypeptides or polynucleotides into plant cells and subsequent insertion into the plant genome include microinjection (Crossway et al. (1986) Biotechniques 4: 320-334), electroporation (Riggs et al. (1986) Proc. Natl. Acad. Sci. USA 83: 5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3: 2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6: 923-926); and Lec1 transformation (WO 00/28058). Also see Weissinger et al. (1988) Ann. Rev. Genet. 22: 421-477; Sanford et al. (1987) Particulate Science and Technology 5: 27-37 (onion); Christou et al. (1988) Plant Physiol. 87: 671-674 (soybean); McCabe et al. (1988) Bio/Technology 6: 923-926 (soybean); Finer and McMullen (1991) In Vitro Cell Dev. Biol. 27P: 175-182 (soybean); Singh et al. (1998) Theor. Appl. Genet. 96: 319-324 (soybean); Datta et al. (1990) Biotechnology 8: 736-740 (rice); Klein et al. (1988) Proc. Natl. Acad. Sci. USA 85: 4305-4309 (maize); Klein et al. (1988) Biotechnology 6: 559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91: 440-444 (maize); Fromm et al. (1990) Biotechnology 8: 833-839 (maize); Hooykaas-Van Slogteren et al. (1984) Nature (London) 311: 763-764; Bowen et al., U.S. Pat. No. 5,736,369 (cereals); Bytebier et al. (1987) Proc. Natl. Acad. Sci. USA 84: 5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9: 415-418 and Kaeppler et al. (1992) Theor. Appl. Genet. 84: 560-566 (whisker-mediated transformation); D'Halluin et al. (1992) Plant Cell 4: 1495-1505 (electroporation); Li et al. (1993) Plant Cell Reports 12: 250-255 and Christou and Ford (1995) Annals of Botany 75: 407-413 (rice); Osjoda et al. (1996) Nature Biotechnology 14: 745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5: 81-84. These plants may then be grown, and either pollinated with the same transformed strain or different strains, and the resulting progeny having the desired phenotypic characteristic identified. Two or more generations may be grown to ensure that the desired phenotypic characteristic is stably maintained and inherited and then seeds harvested to ensure that stable transformants exhibiting the desired phenotypic characteristic have been achieved. In this manner, the present invention provides transformed seed (also referred to as “transgenic seed”) having a nucleotide construct of the invention, for example, a cassette of the invention, stably incorporated into their genome.

As used herein, the term “plant” includes plant cells, plant protoplasts, plant cell tissue cultures from which maize plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art, including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Thus, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

In other embodiments, the polynucleotides of the invention may be introduced into plants by contacting plants with a virus or viral nucleic acids. Generally, such methods involve incorporating a nucleotide construct of the invention within a viral DNA or RNA molecule. It is recognized that an MIK of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191; 5,889,190; 5,866,785; 5,589,367; 5,316,931, and Porta et al. (1996) Molecular Biotechnology 5: 209-221; herein incorporated by reference.

The use of the term polynucleotides herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

The promoter nucleotide sequences and methods disclosed herein are useful in regulating expression of any heterologous nucleotide sequence in a host plant in order to vary the phenotype of a plant. Because the Lpa3 promoter provides embryo-preferred expression of operably linked coding regions, the Lpa3 promoter finds particular use in altering gene expression in or in altering the content of embryos, for example, maize embryos.

Various changes in phenotype are of interest including modifying the fatty acid composition in seeds, altering the amino acid content of seeds, altering a seed's pathogen defense mechanism, and the like. These results can be achieved by providing expression of heterologous products or increased expression of endogenous products in embryos. Alternatively, the results can be achieved by providing for a reduction of expression of one or more endogenous products, particularly enzymes or cofactors in the seed. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered by genetic engineering in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165: 99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502); corn (Pedersen et al. (1986) J. Biol Chem. 261: 6279; Kirihara et al. (1988) Gene 71: 359); and rice (Musumura et al. (1989) Plant Mol. Biol. 12: 123). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48: 109, and the like.

Genes encoding disease resistance traits include detoxification genes, such as against fumonisin (U.S. Pat. No. 5,792,931); avirulence (avr) and disease resistance (R) genes (Jones et al. (1994) Science 266: 789; Martin et al. (1993) Science 262:1432; and Mindrinos et al. (1994) Cell 78: 1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170: 5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### EXPERIMENTAL

### Example 1

### Identification and Characterization of Maize Low Phytic Acid (Lpa) Mutant Plants

A collection of F2 seeds of individual TUSC-mutagenized maize lines was screened for seeds having high inorganic phosphate content using a rapid Pi assay as described below. The TUSC process for selecting Mu insertions in selected genes has been described (see, e.g., Bensen et al. (1995) Plant Cell 7: 75-84; Mena et al. (1996) Science 274: 1537-1540; U.S. Pat. No. 5,962,764, herein incorporated by reference). Candidates identified as producing high-Pᵢ seed were further screened for reduced phytic acid content in mature seeds compared to corresponding wild-type controls. Candidates were crossed with suitable maize and the progeny examined to confirm the mutations and to determine whether the mutations were merely allelic to previously known mutants lpa1 and lpa2. One candidate line was identified as containing a mutation that was non-allelic to both lpa1 and lpa2 and was found to contain a single-locus recessive mutation which was designated lpa3-1. Three additional Mu-insertion alleles of lpa3 were also identified by this screen and were designated lpa3-2, lpa3-3, and lpa3-4.

Lpa3 homozygous mutants have normal seed development, morphology, and germination. The behavior of the mutant was examined in different genetic backgrounds and growth environments, and lpa3 homozygous seeds were found to have phytic acid content that was reduced by 30% to 50% in comparison to corresponding wild-type seeds (see Table 1 below). Mutant lpa3 seeds accumulate inorganic phosphate and myo-inositol but do not accumulate inositol phosphate intermediates. This phenotype contrasts with the phenotype of lpa2 mutants, which accumulate inositol phosphate intermediates, and implies that the Lpa3 gene is involved in the upstream portion of the phytic acid pathway.

Inorganic Phosphate (Pi) Assay

A rapid test was used to assay inorganic phosphate content in kernels. Individual kernels were placed in a 25-well plastic tray and crushed at 2000 psi using a hydraulic press. Two milliliters of 1N H₂SO₄ was added to each sample. The samples were incubated at room temperature for two hours, after which four milliliters of 0.42% ammonium molybdate-1N H₂SO₄:10% ascorbic acid (6:1) was added to each sample. Increased Pᵢ content was signaled by the development of blue color within about 20 minutes. Positive controls included lpa2 mutant kernels, and negative controls included wild-type kernels.

Determination of Phytic Acid and Inorganic Phosphate Content

Dry, mature seeds were assayed for phytic acid and Pᵢ content using modifications of the methods described by Haug and Lantzsch ((1983) J. Sci. Food Agric. 34: 1423-1426, entitled “Sensitive method for the rapid determination of phytate in cereals and cereal products”) and Chen et al. ((1956) Anal. Chem. 28: 1756-1758, entitled “Microdetermination of phosphorus”). Single kernels were ground using a Geno/Grinder2000™ grinder (Sepx CertiPrep®, Metuchen, N.J.). Samples of 25 to 35 mg were placed into 1.5 ml Eppendorf® tubes and 1 ml of 0.4 N HCl was added to the tubes, which were then shaken on a gyratory shaker at room temperature for 3.5 hours. The tubes were then centrifuged at 3,900 g for 15 minutes. Supernatants were transferred into fresh tubes and used for both phytic acid and Pᵢ determinations; measurements were performed in duplicate.

For the phytic acid assay, 35 μl of each extract was placed into wells of a 96-well microtiter plate and then 35 μl of distilled H₂O and 140 μl of 0.02% ammonium iron (III) sulphate-0.2 N HCl were added to each sample. The microtiter plate was covered with a rubber lid and heated in a thermal cycler at 99° C. for 30 minutes, then cooled to 4° C. and kept on an ice water bath for 15 minutes, and then left at room temperature for 20 minutes. The plate was then sealed with sticky foil and centrifuged at 3,900 g at 24° C. for 30 minutes. Eighty μl of each supernatant was placed into wells of a fresh 96-well plate. For absorbance measurements, 120 μl of 1% 2,2′-bipyridine-1% thioglycolic acid solution (10 g 2,2′-bipyridine (Merck® Art. 3098), 10 ml thioglycolic acid (Merck Art. 300) in ddw to 1 liter) was added to each well and absorbance was recorded at 519 nm using a VERSAmax™ microplate reader (Molecular Devices®, Sunnyvale, Calif.). Phytic acid content is presented as phytic acid phosphorus (PAP; see Table 1, below). Authentic phytic acid (Sigma®, P-7660) served as a standard. This phytic acid assay also measures InsP₅ and InsP₄ present in the samples.

Phytic acid was also assayed according to modifications of the methods described by Latta & Eskin (1980) (J. Agric Food Chem. 28: 1313-1315) and Vaintraub & Lapteva (1988) (Analytical Biochemistry 175: 227-230). For this assay, 25 μl of extract was placed into wells of a 96-well microtiter plate; then 275 μl of a solution of 36.3 mM NaOH and 100 μl of Wade reagent (0.3% sulfosalicylic acid in 0.03% FeCl₃.6H₂O) was added to each well. The samples were mixed and centrifuged at 39,000 g at 24° C. for 10 minutes. An aliquot of supernatant (200 μl) from each well was transferred into a new 96-well plate, and absorbance was recorded at 500 nm using a VERSAmax™ microplate reader (Molecular Devices®, Sunnyvale, Calif.).

To determine Pᵢ, 200 μl of each extract was placed into wells of a 96 well microtiter plate. 100 μl of 30% aqueous trichloroacetic acid was then added to each sample and the plates were shaken and then centrifuged at 3,900 g for 10 minutes. Fifty μl of each supernatant was transferred into a fresh plate and 100 μl of 0.42% ammonium molybdate-1N H₂SO₄: 10% ascorbic acid (7:1) was added to each sample. The plates were incubated at 37° C. for 30 minutes and then absorbance was measured at 800 nm. Potassium phosphate was used as a standard. Pᵢ content was presented as inorganic phosphate phosphorus.

Determination of Seed Myo-Inositol

Myo-inositol was quantified in dry, mature seeds and excised embryos. Tissue was ground as described above and mixed thoroughly. 100 milligram samples were placed into 7 ml scintillation vials and 1 ml of 50% aqueous ethanol was added to each sample. The vials were then shaken on a gyratory shaker at room temperature for 1 hour. Extracts were decanted through a 0.45 μm nylon syringe filter attached to a 1 ml syringe barrel. Residues were re-extracted with 1 ml fresh 50% aqueous ethanol and the second extracts were filtered as before. The two filtrates were combined in a 10×75 mm glass tube and evaporated to dryness in a SpeedVac® microcentrifuge (Savant). The myo-inositol derivative was produced by redissolving the residues in 50 μl of pyridine and 50 μl of trimethylsilyl-imidazole:trimethylchlorosilane (100:1) (Tacke and Casper (1996) J. AOAC Int. 79: 472-475). Precipitate appearing at this stage indicates that the silylation reaction did not work properly. The tubes were capped and incubated at 60° C. for 15 minutes. One milliliter of 2,2,4-trimethylpentane and 0.5 milliliters of distilled water were added to each sample. The samples were then vortexed and centrifuged at 1,000 g for 5 minutes. The upper organic layers were transferred with Pasteur pipettes into 2 milliliter glass autosampler vials and crimp-capped.

Myo-inositol was quantified as a hexa-trimethylsilyl ether derivative using an Agilent® model 5890 gas chromatograph coupled with an Agilent® model 5972 mass spectrometer. Measurements were performed in triplicate. One μl samples were introduced in the splitless mode onto a 30 m×0.25 mm i.d.×0.25 μm film thickness 5MS column (Agilent® Technologies). The initial oven temperature of 70° C. was held for 2 minutes, then increased at 25° C. per minute to 170° C., then increased at 5° C. per minute to 215° C., and finally increased at 25° C. per minute to 250° C. and then held for 5 minutes. The inlet and transfer line temperatures were 250° C. Helium at a constant flow of 1 ml per minute was used as the carrier gas. Electron impact mass spectra from m/z 50-560 were acquired at—70 eV after a 5-minute solvent delay. The myo-inositol derivative was well resolved from other peaks in the total ion chromatograms. Authentic myo-inositol standards in aqueous solutions were dried, derivatized, and analyzed at the same time. Regression coefficients of four-point calibration curves were typically 0.999-1.000.

Pᵢ and myo-inositol may also be quantified as described in Shi et al. (2003) Plant Physiol. 131: 507-515.

Determination of Seed Inositol Phosphates

The presence of significant amounts of inositol phosphates in mature seeds was determined by HPLC according to the Dionex Application Note AN65, “Analysis of inositol phosphates” (Dionex® Corporation, Sunnyvale, Calif.). Tissue was ground and mixed as described above. 500 mg samples were placed into 20 ml scintillation vials and 5 ml of 0.4 M HCl was added to the samples. The samples were shaken on a gyratory shaker at room temperature for 2 hours and then allowed to sit at 4° C. overnight. Extracts were centrifuged at 1,000 g for 10 min and filtered through a 0.45 μm nylon syringe filter attached to a 5 ml syringe barrel. Just prior to HPLC analysis, 600 μl aliquots of each sample were clarified by passage through a 0.22 μm centrifugal filter. A Dionex DX 500 HPLC with a Dionex® model AS3500 autosampler was used. 25 μl samples were introduced onto a Dionex® 4×250 mm OmniPac™ PAX-100 column; Dionex® 4×50 mm OmniPac™ PAX-100 guard and ATC-1 anion trap columns also were used. Inositol phosphates were eluted at 1 ml/min with the following mobile phase gradient: 68% A (distilled water)/30% B (200 mM NaOH) for 4.0 min; 39% A/59% B at 4.1 through 15.0 min; return to initial conditions at 15.1 min. The mobile phase contained 2% C (50% aqueous isopropanol) at all times to maintain column performance. A Dionex® conductivity detector module II was used with a Dionex® ASRS-Ultra II anion self-regenerating suppressor set up in the external water mode and operated with a current of 300 mA. Although quantitative standards were available, InsP₃, InsP₄ and InsP₅ were partially but clearly resolved from each other and InsP₆.

The results of the above assays demonstrated that the lpa3 mutant maize plants have a phenotype of reduced phytic acid, increased myo-inositol, and increased Pᵢ in seeds (Table 1). However, lpa3 seeds did not accumulate inositol phosphate intermediates, in contrast to lpa2 seeds (Table 2).

TABLE 1
Myo-inositol and Phytic Acid Content of lpa3 Mutant Seeds is Altered

| | | Myo-inositol content |
|---|---|---|
| Lpa3 Phenotype | PAP (mg/g) | (μg/g) |
| wildtype (strain 1) | 3.03 +/− 0.25 | 168.11 +/− 18.46 |
| wildtype (strain 2) | 2.66 +/− 0.26 | 105.80 +/− 21.15 |
| lpa3 (strain 1) | 1.49 +/− 0.29 | 210.06 +/− 31.18 |
| lpa3 (strain 2) | 1.25 +/− 0.37 | 260.36 +/− 53.84 |

Measurements of Pᵢ and PAP in dissected strain 1 embryos showed wildtype strain 1 embryos had Pᵢ levels of 0.47+/−0.07 mg/g and PAP levels of 25.22+/−2.32 mg/g, while lpa3 strain 1 embryos had Pᵢ levels of 3.60+/−1.34 mg/g and PAP levels of 11.59+/−0.25 mg/g. Measurements of myo-inositol in dissected embryos of another strain showed wildtype embryos had myo-inositol levels of 335 micrograms/g, whereas lpa3 embryos had levels of 580 micrograms/g.

TABLE 2
Accumulation of Inositol Phosphate Intermediates
in lpa2, lpa3, and Wildtype Seeds

| | InsP6 P | InsP5 P | InsP4 P and | Total InsP P |
|---|---|---|---|---|
| Lpa Phenotype | (mg/g) | (mg/g) | InsP3 P (mg/g) | (mg/g) |
| wildtype (strain 1) | 3.70 | 0.13 | 0 | 3.83 |
| lpa3 (strain 1) | 1.83 | 0 | 0 | 1.83 |
| wildtype (strain 3) | 4.00 | 0.11 | 0 | 4.00 |
| lpa2 (strain 3) | 2.37 | 0.88 | 0.24 | 3.48 |

“0” = undetectable with assay used

Although mutant lpa3 seeds accumulate myo-inositol, no significant differences were detected in several myo-inositol related metabolites, such as phosphoinositides, D-ononitol, D-pinitol, glucuronate, and major cell wall sugars. Mutant lpa3 seeds germinate and develop normally. Previously, it had been shown that overexpressing Ins(3)P synthase in Arabidopsis also resulted in increased myo-inositol content, and similarly, no obvious differences in plant growth or development were observed (Smart and Flores (1997) Plant Mol. Biol. 33: 811-820). However, down-regulating Ins(3)P synthase in potato depleted myo-inositol and resulted in smaller tuber and lower tuber yield, altered leaf morphology, reduced apical dominance, reduced galactinol and raffinose contents, and increased hexose phosphates, sucrose and starch concentration (Keller et al. (1998) Plant J. 16: 403-410). Apparently, elevated myo-inositol content does not adversely affect plants whether the elevation is a result of increased biosynthesis or reduced conversion.

Creation of lpa21lpa3 Double Mutant

The maize lpa2 mutant is defective in an inositol phosphate kinase (ZmIPK). See, e.g., Shi et al. (2003) Plant Physiol. 131: 507-515. This mutation also impairs phytic acid biosynthesis and affects the latter part of the biosynthetic pathway, downstream from ZmMIK. An lpa2/lpa3 double mutant was constructed by crossing lpa2 and lpa3 plants, followed by self-pollination of the F1 plants. Double homozygous seeds (i.e., lpa2/lpa3 seeds) looked normal and germinated like wild-type seeds. Evaluation of phytic acid content of these seeds showed a lower phytic acid content than results from either the lpa2 or lpa3 mutation alone (see results in Table 3). The double homozygous seeds also accumulated inositol phosphate intermediates.

Particularly, homozygous sibling lines of wildtype, lpa2, lpa3 and lpa2/lpa3 genotypes were identified from a segregation population constructed by crossing lpa2 and lpa3 mutant lines followed by two generations of self-pollination. Ten mature seeds from each ear were pooled and assayed for phytic acid content. The reduction in phytic acid content (expressed as phytic acid phosphorus (PAP)) is shown below in Table 3.

TABLE 3
Phytic Acid Reduction in the Seed of lpa2, lpa3, and
lpa2/lpa3 Double Mutants

| Genotype | PAP (mg/g) | PA reduction (%) |
|---|---|---|
| lpa2/lpa3 double mutant | 0.81 ± 0.08 | 66 |
| lpa2 | 1.63 ± 0.10* | 31 |
| lpa3 | 1.33 ± 0.11* | 45 |
| Wildtype | 2.36 ± 0.05* | — |

### Example 2

### Isolation and Characterization of Maize Myo-Inositol Kinase

The Mu-tagged Lpa3 gene was cloned by identifying a PCR product that was present in an amplification from lpa3 genomic DNA but that was missing from an amplification from wildtype genomic DNA. Genomic DNA was extracted from individuals of wildtype and lpa3 plants and digested with the AluI restriction enzyme that recognizes a four-nucleotide sequence and cleaves leaving a blunt end. The digested DNA was ligated to an adaptor, which was constructed by annealing the following oligonucleotides according to instructions provided with the Universal GenomeWalker™ Kit (BD Biosciences Clontech®, Palo Alto, Calif.).:

(SEQ ID NO:8)

5′-PO₄-ACCAGCCC-NH₂-3′,
and

(SEQ ID NO:9)

5′-GTAATACGACTCACTATAGGGCACGCGTGGTCGACGGCCCGGGCTGG
T-3′,

The ligation product was purified using the QIAquick™ PCR Purification Kit (Qiagen®), and used as template DNA for a PCR reaction using the following primers:

(SEQ ID NO:10)

5′-AGAGAAGCCAACGCCAWCGCCTCYATTTCGTC-3′,
and

(SEQ ID NO:11)

5′-GTAATACGACTCACTATAGGGC-3′.

Thermocycling conditions were as follows: 1 cycle of denaturing for 15 seconds at 94° C.; 10 cycles of denaturing for 15 seconds at 94° C.; 1 cycle of annealing and elongating for 135 seconds at 68° C.; 15 cycles of denaturing for 15 sec at 94° C.; 1 cycle of annealing and elongating at 68° C. for 135 seconds plus 5 seconds in each successive cycle; and 1 cycle of elongating for 6 min at 68° C.

The PCR product was diluted 1:50 with distilled water and used as the template for nested PCR with the primer 5′-ACTATAGGGCACGCGTGGT-3′ (SEQ ID NO: 35) and each of the following +2 selective Mu primers:

| | |
|---|---|
| 5′-CTCTTCGTCYATAATGGCAATTATCTCAA-3′; | (SEQ ID NO:12) |
| 5′-CTCTTCGTCYATAATGGCAATTATCTCAT-3′; | (SEQ ID NO:13) |
| 5′-CTCTTCGTCYATAATGGCAATTATCTCAC-3′; | (SEQ ID NO:14) |
| 5′-CTCTTCGTCYATAATGGCAATTATCTCAG-3′; | (SEQ ID NO:15) |
| 5′-CTCTTCGTCYATAATGGCAATTATCTCTA-3′; | (SEQ ID NO:16) |
| 5′-CTCTTCGTCYATAATGGCAATTATCTCTT-3′; | (SEQ ID NO:17) |
| 5′-CTCTTCGTCYATAATGGCAATTATCTCTC-3′; | (SEQ ID NO:18) |
| 5′-CTCTTCGTCYATAATGGCAATTATCTCTG-3′; | (SEQ ID NO:19) |
| 5′-CTCTTCGTCYATAATGGCAATTATCTCCA-3′; | (SEQ ID NO:20) |
| 5′-CTCTTCGTCYATAATGGCAATTATCTCCT-3′; | (SEQ ID NO:21) |
| 5′-CTCTTCGTCYATAATGGCAATTATCTCCC-3′; | (SEQ ID NO:22) |
| 5′-CTCTTCGTCYATAATGGCAATTATCTCCG-3′; | (SEQ ID NO:23) |
| 5′-CTCTTCGTCYATAATGGCAATTATCTCGA-3′; | (SEQ ID NO:24) |
| 5′-CTCTTCGTCYATAATGGCAATTATCTCGT-3′; | (SEQ ID NO:25) |
| 5′-CTCTTCGTCYATAATGGCAATTATCTCGC-3′; | (SEQ ID NO:26) |
| and | |
| 5′-CTCTTCGTCYATAATGGCAATTATCTCGG-3′. | (SEQ ID NO:27) |

The PCR products were analyzed on agarose gels using standard molecular biology techniques, and a band was identified that was present only in lpa3 mutants but not in wild-type plants. This band was cut from the gel and the DNA in the band was purified and the PCR product was sequenced. This partial sequence was used to search a database of ESTs prepared from inbred B73 maize plants. Several overlapping ESTs were identified, including EST ceflf.pk001.f15, which has the nucleotide sequence set forth in SEQ ID NO: 1 and encodes a polypeptide having the amino acid sequence set forth in SEQ ID NO: 2. This polypeptide was determined to have myo-inositol kinase activity and was designated ZmMIK (Zea mays myo-inositol kinase) or Lpa3 (low phytic acid). The Lpa3 protein contains 379 amino acids and has a calculated molecular weight of about 39.9 kiloDaltons and a pI of about 5.2.

Thus, SEQ ID NO: 1 sets forth the cDNA sequence of ZmMIK (Lpa3), and SEQ ID NO: 2 sets forth the amino acid sequence of the ZmMIK (Lpa3) protein. The genomic copy of the Lpa3 gene (set forth in SEQ ID NO:3) includes: the transcriptional regulatory portion, including a promoter which directs embryo-preferred expression (nucleotides 1 to 1379 (SEQ ID NO: 4); see Example 4); exon 1 (nucleotides 1380-2582), which encodes the 5′ untranslated region and N-terminal portion of Lpa3; intron 1 (nucleotides 2583-4067); and exon 2 (nucleotides 4076-4622), which encodes the C-terminal portion of Lpa3 and 3′ untranslated region.

The Lpa3 sequence was used to search an EST database prepared from the tassels of inbred W23 maize plants. This search revealed an EST (SEQ ID NO: 5) that encodes a variant Lpa3 polypeptide (SEQ ID NO: 6, designated “ZmMIKv”) which differs from Lpa3 at positions 71 and 200.

ZmMIK Polypeptides Have Three Conserved Domains

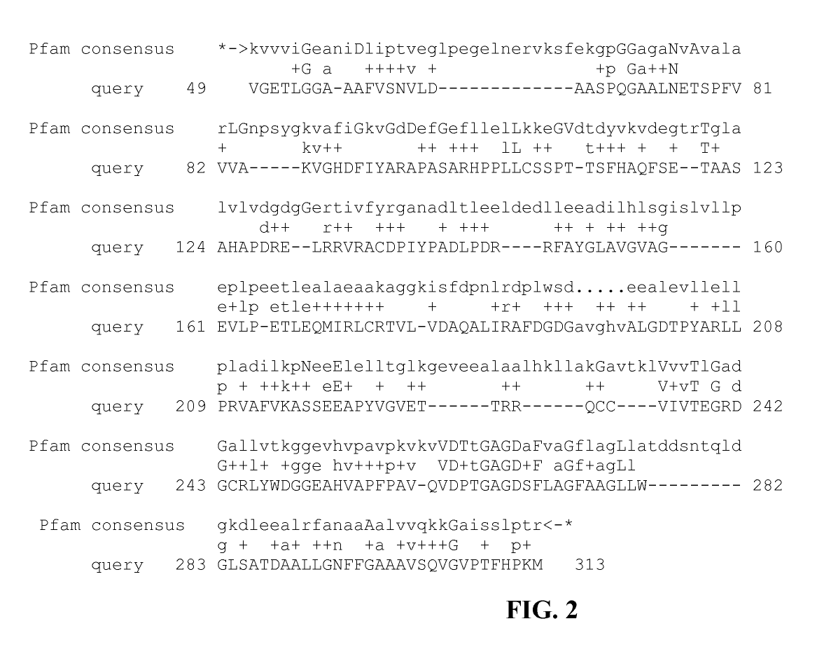

The Lpa3 polypeptide contains consensus features of the pfkB carbohydrate kinase family of proteins. FIG. 2 shows a comparison of Lpa3 with pfam00294, the pfkB family carbohydrate kinase consensus sequence (SEQ ID NO: 7). The sequences were searched and aligned using the bioSCOUT™ software program. The pfkB family includes a variety of carbohydrate and pyrimidine kinases, including, for example, phosphomethylpyrimidine kinase (EC:2.7.4.7), which is part of the synthesis pathway for thiamine pyrophosphate (TPP), an essential cofactor for many enzymes. The pfkB family also includes ribokinase, fructokinase, fructose 1-phosphate kinase, 6-phosphofructokinase isozyme 2 (pfkB), pyridoxal kinase, and adenosine kinase (Wu et al. (1991) J. Bacteriol. 173: 3117-3127). Although none of the known inositol phosphate kinases belongs to the pfkB or related kinase families, the protein sequence alignment when considered together with the lpa3 mutant phenotype suggested that the Lpa3 gene might encode a myo-inositol kinase or a new inositol phosphate kinase.

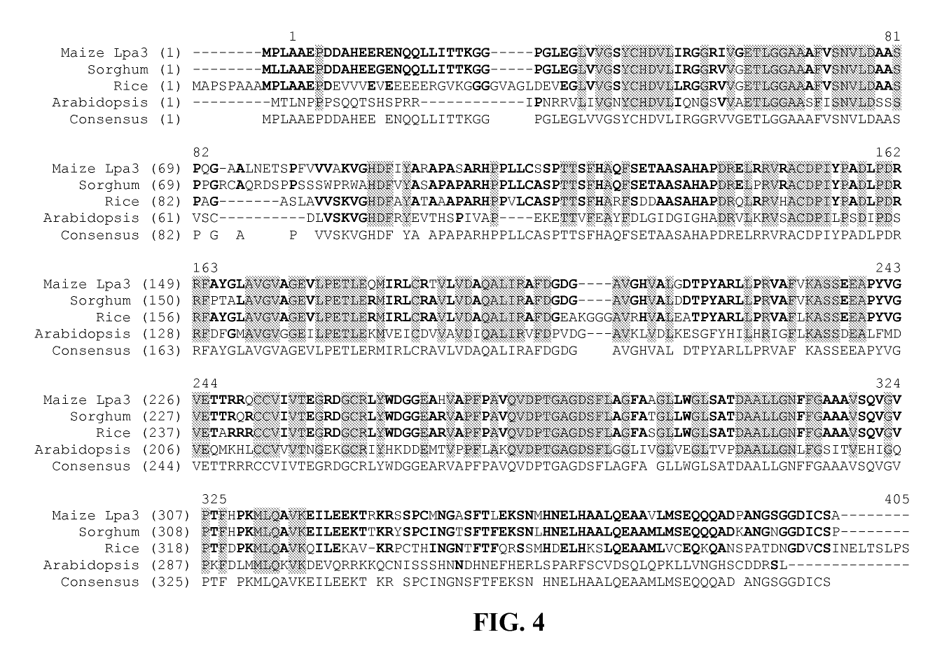

Additional database searches identified similar proteins from other plants (i.e., orthologs). FIG. 4 shows an alignment of the Lpa3 polypeptide (SEQ ID NO: 2) with a rice protein (GenBank Acc. No. AP03418; SEQ ID NO: 28), a sorghum protein (SEQ ID NO: 30), and an Arabidopsis protein (GenBank Acc. No. NP_{—}200681; SEQ ID NO: 29) which also contain consensus features of the pfkB carbohydrate kinase family. The alignment also demonstrates substantial sequence homology of these proteins over their entire length (FIG. 4; consensus sequence is set forth in SEQ ID NO: 41). Accordingly, the invention additionally provides plant proteins comprising this consensus sequence and polynucleotides encoding them.

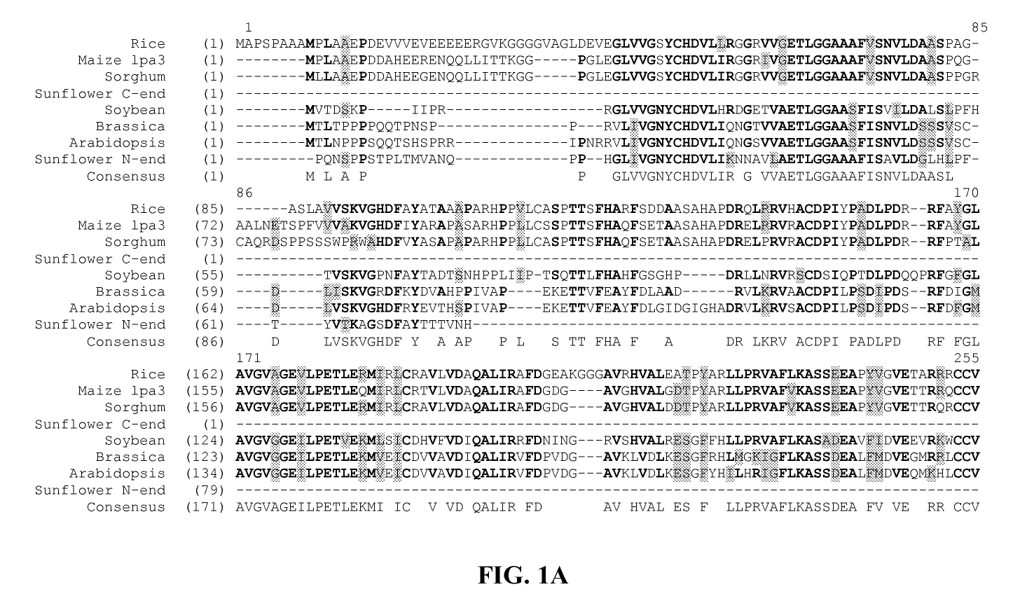

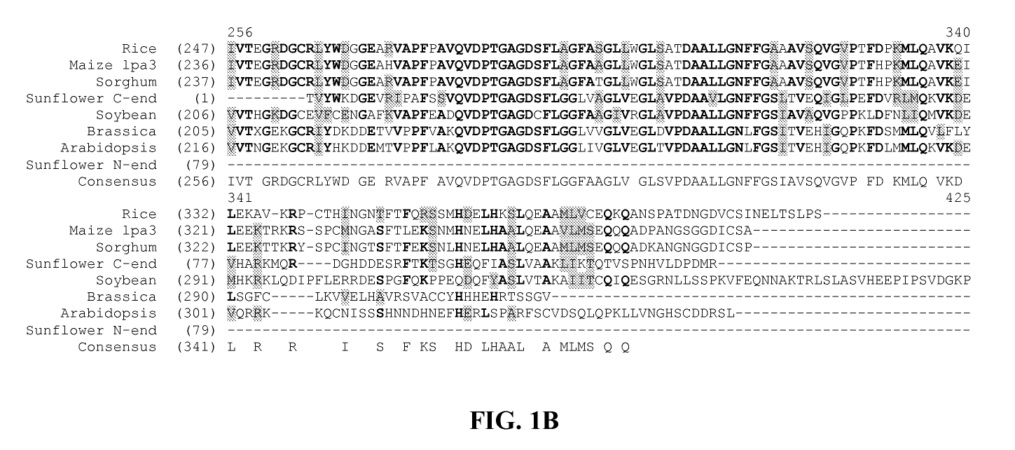

In FIGS. 1A and 1B, the Lpa3 polypeptide sequence (SEQ ID NO: 2), rice protein (SEQ ID NO: 28; GenBank Acc. No. AAP03418), and Arabidopsis pfkB family carbohydrate kinase (SEQ ID NO: 29; GenBank Acc. No. NP_{—}200681) are aligned with the Sorghum bicolor protein (SEQ ID NO: 30; ORF from sorghum BAC genomic sequence in GenBank Acc. No. AF124045), a Brassica oleracea protein (SEQ ID NO: 31, assembled from GenBank Acc. Nos. BH473-483, BH553276, and BH709390), a sunflower protein (N-terminal sequence (SEQ ID NO: 32) from EST QHJ9H03.yg.ab1, GenBank Acc. No. BU036303; C-terminal sequence (SEQ ID NO: 33) from EST DH0AG10ZH05RM1, GenBank Acc. No. CD857535), and a soybean protein (SEQ ID NO: 34) from Pioneer/DuPont EST src3c.pk028.p5.fis. This alignment revealed an overall consensus sequence (SEQ ID NO: 40) and three conserved domains which are designated A, B, and C (diagrammed in FIG. 3) and which have the following consensus sequences:

Domain A (SEQ ID NO:36):
L(V/I)VGXYCHDVL(I/L)(R/K)XGX(V/I)(V/L)(A/G)ETLGGAA
(A/S)F(I/V)SX(V/I)LD
Domain B (SEQ ID NO:37):
RXLXRVXACDPIXP(A/S)DLPDXRFXX(G/A)(L/M)AVGV(A/G)GE
(V/I)LPETLEXM(V/I)X(L/I)CXXVXVDXQALIRXFD
Domain C (SEQ ID NO:38):
QVDPTGAGDSFL(G/A)GXXXG(L/I)(V/L)XGLXXXDAA(L/V)LGNF
FG(S/A)

where “X” indicates any amino acid. The portion of Domain C italicized above and set forth separately in SEQ ID NO: 39 is also conserved in the pfkB carbohydrate kinase family. Accordingly, the invention additionally provides plant proteins comprising these consensus sequences and domains as well as polynucleotides encoding them.
Expression and Purification of ZmMIK

The Lpa3 gene product was expressed as a glutathione-S-transferase (GST) fusion protein in E. coli and purified. A single colony of E. coli strain DH5a containing a GST-tagged Lpa3 construct in an expression vector was cultured overnight at 37° C. in LB medium containing ampicillin (“LB+Amp”). The overnight culture was diluted 1:10 with fresh LB +Amp medium and incubated at 37° C. with vigorous agitation until the A600 reading of the culture was in the range of 0.6 to 2 OD units. GST fusion protein expression was induced by the addition of IPTG to the culture to a final concentration of 50 μM and the cultures were incubated at 37° C. with agitation for an additional 3 hours.

Bacteria were harvested by centrifugation at 7,700 g for 10 min at 4° C. Pellets were resuspended in ice-cold bacterial lysis buffer (50 mM Tris-HCl (pH 7.4), 100 mM NaCl, 100 μM phenylmethylsulfonyl fluoride), and lysed on ice by sonication. The lysate was clarified by centrifugation at 12,000 g for 10 min at 4° C. The Lpa3-GST proteins were affinity purified by batch absorption to glutathione Sepharose® 4B gel slurry with a 45 minute incubation at 4° C. with gentle shaking. The beads were washed four times with lysis buffer and twice with phosphate buffered saline according to the manufacturer's instructions (Amersham Biosciences Corporation, Piscataway, N.J.). Lpa3-GST protein was eluted with 10 mM reduced glutathione in 50 mM Tris-HCl (pH 8.0), 100 mM NaCl (200 μl buffer for every 500 ml of cell culture). After elution, glycerol was added to a final concentration of 50% and the proteins were stored at −20° C.

MIK Activity and Substrate Specificity Assays

myo-inositol kinase activities were assayed according to Wilson and Majerus ((1996) J. Biol. Chem. 271: 11904-11910), with modifications as indicated below. Each assay was performed in 25 μl of assay mixture, which contained 20 mM HEPES (pH 7.2), 6 mM MgCl₂, 10 mM LiCl, 1 mM DTT, 40 μM myo-inositol, 40 μM ATP, 0.5 μl γ-³²P-ATP (3000 Ci/mmol) and 5 μl enzyme. The reaction mixture was incubated at 30° C. for 30 minutes and the reaction was stopped by the addition of 2.8 μl stopping solution (3M HCl, 2M KH₂PO₄). A 1 μl sample from each reaction was separated on a thin layer chromatography plate precoated with high-performance cellulose (Merck®) using 1-propanol:25% ammonia solution:water (5:4:1; see Hatzack and Rasmussen (1999) J. Chromat. B 736: 221-229). After separation, the TLC plate was air-dried at 70° C., wrapped in plastic wrap and exposed to X-ray film to detect the ³²P-labelled reaction products. The substrate specificity of ZmMIK was tested by using scyllo-inositol and myo-inositol phosphates in addition to myo-inositol. Myo-inositol phosphate substrates tested under the same conditions included Ins(1)P, Ins(2)P, Ins(3)P, Ins(4)P, Ins(1,4)P₂, Ins(2,4)P₂ and Ins(4,5)P₂.

Results showed that the Lpa3 protein phosphorylated the myo-inositol substrate to produce a ³²P-labelled product that comigrated with myo-inositol mono-phosphate on high performance cellulose TLC plates; that is, the Lpa3 protein exhibited myo-inositol kinase activity. The Lpa3 protein also used scyllo-inositol as a substrate in the in vitro assay. However, the Lpa3 protein has no kinase activity on any of the inositol phosphates tested, including Ins(1)P, Ins(2)P, Ins(3)P, Ins(4)P, Ins(1,4)P₂, Ins(2,4)P₂ and Ins(4,5)P₂. These results demonstrate that Lpa3 protein has myo-inositol kinase activity and provide the first example of a myo-inositol kinase gene cloned from any organism.

Further, results showed that the ZmMIK protein phosphorylates myo-inositol to produce D/L-Ins(3)P, D/L-Ins(4)P and Ins(5)P. This production of multiple products by ZmMIK and the defects of mutant lpa3 plants in phytic acid biosynthesis indicates that phytic acid biosynthesis in developing seeds employs multiple routes. The products were confirmed to be inositol monophosphates by treatment with bovine inositol monophosphatase (Sigma® 1-0274), which completely removed the products. Two of the inositol monophosphates were identified based on their co-elution with authentic standards and their mass spectra; however, this method was unable to distinguish Ins(1)P from its enantiomer Ins(3)P or to distinguish Ins(4)P from its enantiomer Ins(6)P. Because no ZmMIK product co-eluted with the authentic Ins(2)P standard, the third inositol monophosphate product must be Ins(5)P; however, this product accounted for only a small proportion of the ZmMIK products. Ins(3)P was purchased from Matreya, Inc. (State College, Pa.); myo-inositol, scyllo-inositol, Ins(1)P, Ins(2)P, Ins(4)P, Ins(1,4)P₂, Ins(4,5)P₂ and bovine brain inositol monophosphatase were obtained from Sigma (St. Louis, Mo.).

While these experiments were conducted using purified GST-ZmMIK fusion protein, the same results were obtained when the GST tag was removed from the fusion protein by thrombin digestion.

The enzymatic activity of the ZmMIK protein contrasts with the activity of MIK purified from germinating wheat seeds, which was found to produce Ins(3)P (Loewus et al. (1982) Plant Physiol. 70: 1661-1663).

### Example 3

### Stacking Lpa3 with Other Inositol Phosphate Kinase Genes

By “stacking” (i.e., transforming a plant with) constructs designed to reduce or eliminate the expression of Lpa3 and other proteins, it is expected that the reduction of phytic acid and increase in available phosphorus will be enhanced in comparison to plants transformed with constructs designed to reduce or eliminate the expression of Lpa3 alone. Accordingly, four expression cassettes were prepared making use of inverted repeat constructs known as Inverted Repeats Without Terminators, or “IRNTs.” The first and second portion of such constructs self-hybridize to produce a hairpin structure which can suppress expression of the relevant endogenous gene. Expression cassettes 1-4 below each contain an IRNT (“Lpa3 IRNT”) that can suppress endogenous Lpa3 gene expression. This Lpa3 IRNT includes two portions of an Lpa3 inverted repeat surrounding the Adh1 gene intron. In some embodiments, the IRNT comprises substantially the entire Lpa3 cDNA sequence, whereas in other embodiments, the IRNT comprises the entire Lpa3 cDNA but only about 200 nucleotides of the complementary sequence. Expression cassettes 2, 3, and 4 each contain an additional IRNT that can suppress expression of IPPK, ITPK-5, and MI1PS3, respectively. “Glb1” indicates the globulin 1 promoter, and “Ole” indicates the oleosin promoter. Each expression cassette is provided in a plasmid which contains additional useful features.

1) Glb1::Lpa3 IRNT

2) Ole::Lpa3 IRNT+Glb1::IPPK IRNT

3) Glb1::Lpa3 IRNT+Ole::ITPK-5 IRNT

4) Ole::Lpa3 IRNT+Glb1::MIIPS3 IRNT

Design of these plasmids was conducted in view of earlier experiments in which suppression of milps genes was used to produce strong low phytate and high Pi transgenic plants; however, the seeds of these plants had poor germination. It was determined that the myo-inositol content of the seeds of these plants was reduced dramatically and likely contributed to the poor germination. It is hypothesized that suppressing MIK could rescue plants in which mi1ps is also suppressed, which would make possible a further reduction in phytate content and an increase in available phosphorus in seeds.

The plasmids can be inserted into Agrobacterium vectors and used to transform maize cells. Sample protocols for creation of Agrobacterium strains harboring a plasmid are described, for example, in Lin (1995) in Methods in Molecular Biology, ed. Nickoloff, J. A. (Humana Press, Totowa, N.J.). Successful transformation can be verified by restriction analysis of the plasmid after transformation back into E. coli DH5α cells.

### Example 4

### Characterization of Maize Lpa3 Promoter

The 5′ upstream portion of the Lpa3 gene (SEQ ID NO: 4; nucleotides 1 to 1379 of SEQ ID NO: 3) was examined for transcriptional regulatory activity using Lynx™ expression profiling. Lynx™ gene expression profiling technology utilizes massively parallel signature sequence (MPSS; see Brenner et al. (2000) Nature Biotechnology 18: 630-634; Brenner et al. (2000) Proc. Nat'l. Acad. Sci. USA 97: 1665-1670). MPSS generates 17-mer sequence tags of millions of cDNA molecules, which are cloned on microbeads. The technique provides an unprecedented depth and sensitivity of mRNA detection, including messages expressed at very low levels. The ZmMIK gene showed the highest levels of expression in embryos, with a mean of 140 ppm, but its expression in endosperm, as well as in vegetative tissues, is less than 25 ppm. As a reference, the expression level of the oleosin gene in the embryo is about 30,000 ppm and the expression level of the globulin 1 gene is about 3,000 ppm; therefore, the ZmMIK expression levels are relatively low. The embryo-preferred ZmMIK expression pattern was confirmed by Northern analysis of mRNA prepared from developing seeds and vegetative tissues. The Northern analysis confirmed that the Lpa3 gene is expressed in the embryo at 15, 22, and 29 days after pollination (DAP). Lpa3 expression was not detected in roots, leaves, or whole kernels 7 DAP nor in endosperm at 15, 22, and 29 DAP. These results are consistent with what is known about phytic acid synthesis and accumulation in seeds. In maize seeds, phytic acid is found predominantly in embryo and aleurone cells, while only trace phytic acid is found in endosperm. These results also indicate that the Lpa3 promoter is a tissue-preferred promoter that directs expression of the Lpa3 coding region in the embryo at levels of about 100 ppm to 350 ppm between 20 and 45 days after pollination (DAP).

### Example 5

### Production of Lpa3 Transgenic Plants Using Agrobacterium-Mediated Transformation

For Agrobacterium-mediated transformation of maize with an Lpa3 construct of the invention, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the Lpa3 construct to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite™ (added after bringing to volume with D-I H₂O); and 8.5 mg/l silver nitrate (added after sterilizing the medium and cooling to room temperature). Selection medium (560R) comprises 4.0 g/l N6 basal salts (Sigma® C-1416), 1.0 ml/l Eriksson's Vitamin Mix (1000× Sigma®-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite™ (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (Gibco® 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite™ (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (Gibco® 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l Bacto-agar (added after bringing to volume with polished D-I H₂O), sterilized and cooled to 60° C.

### Example 6

### Production of Lpa3 Transgenic Plants Using Soybean Embryo Transformation

Soybean embryos are bombarded with a plasmid containing an Lpa3 construct as follows. A seed-specific expression cassette composed of the promoter and transcription terminator from the gene encoding the beta subunit of the seed storage protein phaseolin from the bean Phaseolus vulgaris (Doyle et al. (1986) J. Biol. Chem. 261: 9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites NcoI (which includes the ATG translation initiation codin), SmaI, KpnI, and XbaI. The entire cassette is flanked by HindIII sites.

To induce somatic embryos, cotyledons 3-5 mm in length dissected from surface-sterilized, immature seeds of the soybean cultivar A2872 are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker at 150 rpm at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327: 70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313: 810-812), the hygromycin phosphotransferase gene from plasmid pJR225 (from E. coli; Gritz et al. (1983) Gene 25: 179-188), and the 3′ region of the nopaline synthase gene from the T-DNA of the Ti plasmid of Agrobacterium tumefaciens. The expression cassette comprising the Lpa3 construct operably linked to the CaMV 35S promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm Petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 7

### Production of Lpa3 Transgenic Plants Using Brassica napus Seed Transformation

Brassica napus seeds are transformed using a transformation and regeneration protocol modified from Mehra-Palta et al. (1991), “Genetic Transformation of Brassica napus and Brassica rapa,” in Proc. 8^{th} GCIRC Congr., ed. McGregor (University Extension Press, Saskatoon, Sask., Canada), pp. 1108-1115 and Stewart et al. (1996), “Rapid DNA Extraction From Plants,” in Fingerprinting Methods Based on Arbitrarily Primed PCR, Micheli and Bova, eds. (Springer, Berlin), pp. 25-28. See Cardoza and Stewart (2003) Plant Cell Rep. 21: 599-604.

Seeds are surface-sterilized for 5 minutes with 10% sodium hypochlorite with 0.1% Tween™ added as a surfactant, rinsed for one minute with 95% ethanol, and then washed thoroughly with sterile distilled water. Seeds are germinated on MS basal medium (Murashige and Skoog (1962) Physiol. Plant 15: 473-497) containing 20 g/liter sucrose and 2 g/liter Gelrite™. Hypocotyls are excised from 8- to 10-day-old seedlings, cut into 1-cm pieces, and preconditioned for 72 hours on MS medium supplemented with 1 mg/liter 2,4-D (2,4-dichlorophenoxy acetic acid) and containing 30 g/liter sucrose and 2 g/liter Gelrite™.

Agrobacterium containing a plasmid comprising an Lpa3 construct of the invention is grown overnight in liquid LB medium to an OD₆₀₀ of 0.8, pelleted by centrifugation, and resuspended in liquid callus induction medium containing acetosyringone at a final concentration of 0.05 mM. Agrobacterium is then cocultivated with the preconditioned hypocotyl segments for 48 hours on MS medium with 1 mg/liter 2,4-D. After the cocultivation period, explants are transferred to MS medium containing 1 mg/liter 2,4-D, 400 mg/liter timentin, and 200 mg/liter kanamycin to select for transformed cells. After 2 weeks, in order to promote organogenesis, the explants are transferred to MS medium containing 4 mg/liter BAP (6-benzylaminopurine), 2 mg/liter zeatin, 5 mg/liter silver nitrate, antibiotics selective for the transformation construct, 30 g/liter sucrose, and 2 g/liter Gelrite™. After an additional 2 weeks, in order to promote shoot development, tissue is transferred to MS medium containing 3 mg/liter BAP, 2 mg/liter zeatin, antibiotics, 30 g/liter sucrose, and 2 g/liter Gelrite™. Shoots that develop are transferred for elongation to MS medium containing 0.05 mg/liter BAP, 30 g/liter sucrose, antibiotics, and 3 g/liter Gelrite™. Elongated shoots are then transferred to root development medium containing half-strength MS salts, 10 mg/liter sucrose, 3 g/liter Gelrite™, 5 mg/liter IBA (indole-3-butyric acid), and antibiotics. All cultures are maintained at 25° C.+/−2° C. in a 16-hour light/8-hour dark photoperiod regime with light supplied by cool white daylight fluorescent lights. The rooted shoots are transferred to soil and grown under the same photoperiod regime at 20° C. in a plant growth chamber.

Transformation of plants with the Lpa3 construct is confirmed using PCR of DNA extracted from putative transgenic plants.

### Example 8

### Variants of Lpa3

A. Variant Nucleotide Sequences of Lpa3 (SEQ ID NO: 1) That Do Not Alter the Encoded Amino Acid Sequence

The Lpa3 nucleotide sequence set forth in SEQ ID NO: 1 is used to generate variant nucleotide sequences having the nucleotide sequence of the open reading frame with about 70%, 76%, 81%, 86%, 92%, and 97% nucleotide sequence identity when compared to the starting unaltered ORF nucleotide sequence of SEQ ID NO: 1. In some embodiments, these functional variants are generated using a standard codon table. In these embodiments, while the nucleotide sequence of the variant is altered, the amino acid sequence encoded by the open reading frame does not change.

B. Variant Amino Acid Sequences of Lpa3

Variant amino acid sequences of Lpa3 are generated. In this example, one amino acid is altered. Specifically, the open reading frame set forth in SEQ ID NO: 2 is reviewed to determined the appropriate amino acid alteration. The selection of the amino acid to change is made by consulting the protein alignment (with the other orthologs and other gene family members from various species). See FIGS. 3, 4, 5, and 6. An amino acid is selected that is deemed not to be under high selection pressure (not highly conserved) and which is rather easily substituted by an amino acid with similar chemical characteristics (i.e., similar functional side-chain). Using the alignments set forth in FIGS. 3, 4, 5, and/or 6, an appropriate amino acid can be changed. Variants having about 70%, 75%, 80%, 85%, 90%, 95%, and 97% nucleic acid sequence identity to SEQ ID NO: 2 are generated using this method.

C. Additional Variant Amino Acid Sequences of Lpa3

In this example, artificial protein sequences are created having about 80%, 85%, 90%, 95%, and 97% identity relative to the reference protein sequence. This latter effort requires identifying conserved and variable regions from the alignments set forth in FIGS. 3, 4, 5, and/or 6 and then the judicious application of an amino acid substitutions table. These parts will be discussed in more detail below.

Largely, the determination of which amino acid sequences are altered is made based on the conserved regions among MIKs. See FIGS. 3, 4, 5, and 6. It is recognized that conservative substitutions can be made in the conserved regions below without altering function. In addition, one of skill will understand that functional variants of the Lpa3 sequence of the invention can have minor non-conserved amino acid alterations in the conserved domain.

Artificial protein sequences are then created that are different from the original in the intervals of 80-85%, 85-90%, 90-95%, and 95-100% identity. Midpoints of these intervals are targeted, with liberal latitude of plus or minus 1%, 2%, or 3%, for example. The amino acids substitutions will be effected by a custom Perl script. The substitution table is provided below in Table 4.

TABLE 4
Substitution Table

| | Strongly | | |
|---|---|---|---|
| | Similar and | Rank of | |
| Amino | Optimal | Order to | |
| Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C, and P are not changed in any circumstance. The changes will occur with isoleucine first, sweeping N-terminal to C-terminal, then leucine, and so on down the list until the desired target of percent change is reached. Interim number substitutions can be made so as not to cause reversal of changes. The list is ordered 1-17, so start with as many isoleucine changes as needed before leucine, and so on down to methionine. Clearly, many amino acids will in this manner not need to be changed. Changes between L, I, and V will involve a 50:50 substitution of the two alternate optimal substitutions.

The variant amino acid sequences are written as output. Perl script is used to calculate the percent identities. Using this procedure, variants of Lpa3 are generated having about 82%, 87%, 92%, and 97% amino acid identity to the starting unaltered ORF nucleotide sequence of SEQ ID NO: 1.

### Example 9

### Pedigree Breeding

Pedigree breeding starts with the crossing of two genotypes, such as a transformed (i.e., transgenic) inbred line and one other elite inbred line having one or more desirable characteristics that is lacking or which complements the first transgenic inbred line. If the two original parents do not provide all the desired characteristics, other sources can be included in the breeding population. In the pedigree method, superior segregating plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection is practiced: F1→F2; F2→F3; F3→F4; F4→F5, etc. After a sufficient amount of inbreeding, successive filial generations will serve to increase seed of the developed inbred. Preferably, the inbred line comprises homozygous alleles at about 95% or more of its loci.

In addition to being used to create a backcross conversion, backcrossing can also be used in combination with pedigree breeding to modify a transgenic inbred line and a hybrid that is made using the transgenic inbred line. Backcrossing can be used to transfer one or more specifically desirable traits from one line, the donor parent, to an inbred called the recurrent parent, which has overall good agronomic characteristics yet lacks that desirable trait or traits.

Therefore, an embodiment of this invention is a method of making a backcross conversion of a maize transgenic inbred line containing an Lpa3 construct or a mutation such as lpa3-1, comprising the steps of crossing a plant of an elite maize inbred line with a donor plant comprising a mutant gene or transgene conferring a desired trait, selecting an F1 progeny plant comprising the mutant gene or transgene conferring the desired trait, and backcrossing the selected F1 progeny plant to a plant of the elite maize inbred line. This method may further comprise the step of obtaining a molecular marker profile of the elite maize inbred line and using the molecular marker profile to select for a progeny plant with the desired trait and the molecular marker profile of the maize elite inbred line. In the same manner, this method may be used to produce an F1 hybrid seed by adding a final step of crossing the desired trait conversion of the elite maize inbred line with a different maize plant to make F1 hybrid maize seed comprising a mutant gene or transgene conferring the desired trait.

Recurrent Selection and Mass Selection

Recurrent selection is a method used in a plant breeding program to improve a population of plants. The method entails individual plants cross-pollinating with each other to form progeny. The progeny are grown and superior progeny are selected by any number of selection methods, which include individual plant, half-sib progeny, full-sib progeny, selfed progeny and topcross yield evaluation. The selected progeny are cross-pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

Mutation Breeding

Mutation breeding is one of many methods that could be used to introduce new traits into a particular maize inbred line. Mutations that occur spontaneously or are artificially induced can be useful sources of variability for a plant breeder. The goal of artificial mutagenesis is to increase the rate of mutation for a desired characteristic. Mutation rates can be increased by many different means. Such means include: temperature; long-term seed storage; tissue culture conditions; radiation such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm); genetic means such as transposable elements or DNA damage repair mutations; chemical mutagens (such as base analogues (5-bromo-uracil); and related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques, such as backcrossing. Details of mutation breeding can be found in Fehr (1993) “Principals of Cultivar Development” (Macmillan Publishing Company), the disclosure of which is incorporated herein by reference. In addition, mutations created in other lines may be used to produce a backcross conversion of a transgenic elite line that comprises such mutation.

### Example 10

### Gene Silencing with the Lpa3 Promoter

The promoter of a target gene (e.g., Lpa3) is inactivated by introducing into a plant an expression cassette comprising a promoter and an inverted repeat of fragments of the Lpa3 promoter. For example, an expression cassette may be created that comprises the Ole promoter operably linked to an inverted repeat comprising fragments of the Lpa3 promoter that are approximately 200 bp in length and that are separated by the Adh1 intron. The Lpa3 promoter fragments may be selected from a portion of the promoter which is rich in CpG islands, such as, for example, the 3′ portion of the Lpa3 promoter. The sequence of the Lpa3 promoter is set forth in SEQ ID NO: 4 and in nucleotides 1-1379 of SEQ ID NO: 3. The expression cassette is used to transform a plant, which is then assayed for lack of expression of the Lpa3 gene. While the invention is not bound by any particular mechanism of operation, the method is thought to produce a small RNA molecule which recognizes the native promoter of the target gene and leads to methylation and inactivation (i.e., gene silencing) of the native promoter. Consequently, the gene associated with the promoter is not expressed. This trait is heritable and cosegregates with the transgenic construct.

### Example 11

### Transgenic Maize Seeds Have Reduced Phytic Acid Content

Two expression cassettes were constructed to provide cosuppression of an MIK. These expression cassettes (designated plasmids P86 and P20) were made using MIK polynucleotide fragments. Each expression cassette contained an inverted repeat of an MIK polynucleotide such that the first and second portions self-hybridize to produce a hairpin structure that can suppress expression of the relevant endogenous gene (e.g., Lpa3). Between the two fragments of the inverted repeat was an intron that helps to form the loop portion in the hairpin structure. Transcription of the MIK hairpin RNA was driven by the oleosin promoter in plasmid P20 and by the Glb 1 promoter in plasmid P86; neither construct has a terminator. In addition, plasmid P86 contained a second set of fragments similar to that described above for MIK comprising a first and second portion of the IPPK gene in which the second portion was an inverted fragment of the first portion. Transcription of this IPPK hairpin RNA in plasmid P86 was driven by the Glb1 promoter.

Plasmids P20 and P86 were used to produce transgenic maize using protocols described in Example 1. Transgenic T1 seeds were screened for elevated Pᵢ content using a rapid Pᵢ assay, and quantitative analysis of phytic acid was also performed. The results of these assays demonstrated that cosuppression of MIK expression resulted in a decrease in phytic acid content and an increase in Pᵢ in the transgenic seeds (see Table 5).

TABLE 5
Maize Plants Transformed with an MIK Hairpin Expression
Cassette Produced Transgenic Seeds with Reduced Phytic Acid Content

| | CS K | Wt K | PAP |
|---|---|---|---|
| Event | (mg/g) | (mg/g) | reduction |
| Plasmid 20 | | | |
| 27-7 | 0.71 | 1.32 | 46% |
| 97-1 | 0.82 | 1.28 | 36% |
| 01-4 | 1.17 | 1.92 | 39% |
| 86-7 | 0.90 | 1.55 | 42% |
| 72-7 | 1.22 | 1.81 | 32% |
| 26-7 | 1.12 | 1.89 | 41% |
| Plasmid 86 | | | |
| 36-6 | 1.12 | 1.69 | 34% |
| 34-2 | 1.11 | 1.85 | 40% |

Wt K = wild-type kernels in a segregation ear;
CS K = cosuppression kernels in a segregation ear;
PAP = phytic acid phosphorus

As indicated in the table legend, “Wt K” were kernels in a segregation ear without the MIK transgene and “CS K” were the kernels in the same segregation ear that did contain the MRP transgene. The PAP values in Table 4 were measured according to modifications (described in Example 1) of the methods taught by Haug and Lantzsch (1983) J. Sci. Food Agric. 34: 1423-1426.

### Example 12

### Production of Transgenic Sorghum

The promoter construct prepared in Example 10 is used to transform sorghum according to the teachings of U.S. Pat. No. 6,369,298. Briefly, a culture of Agrobacterium is transformed with a vector comprising an expression cassette containing the promoter construct prepared in Example 10. The vector also comprises a T-DNA region into which the promoter construct is inserted. General molecular techniques used in the invention are provided, for example, by Sambrook et al. (eds.) Molecular Cloning: A Laboratory Manual, 1989, Cold Spring Harbor Laboratory Press, Cold Spring Harbor, N.Y.

Immature sorghum embryos are obtained from the fertilized reproductive organs of a mature sorghum plant. Immature embryos are aseptically isolated from the developing kernel at about 5 days to about 12 days after pollination and held in sterile medium until use; generally, the embryos are about 0.8 to about 1.5 mm in size.

The Agrobacterium-mediated transformation process of the invention can be broken into several steps. The basic steps include: an infection step (step 1); a co-cultivation step (step 2); an optional resting step (step 3); a selection step (step 4); and a regeneration step (step 5). In the infection step, the embryos are isolated and the cells contacted with the suspension of Agrobacterium.

The concentration of Agrobacterium used in the infection step and co-cultivation step can affect the transformation frequency. Very high concentrations of Agrobacterium may damage the tissue to be transformed, such as the immature embryos, and result in a reduced callus response. The concentration of Agrobacterium used will vary depending on the Agrobacterium strain utilized, the tissue being transformed, the sorghum genotype being transformed, and the like. Generally a concentration range of about 0.5×10⁹ cfu/ml to 1×10⁹ cfu/ml will be used.

The embryos are incubated with the suspension of Agrobacterium about 5 minutes to about 8 minutes. This incubation or infection step takes place in a liquid solution that includes the major inorganic salts and vitamins of N6 medium (referred to as “N6 salts,” or medium containing about 463.0 mg/l ammonium sulfate; about 1.6 mg/l boric acid; about 125 mg/l calcium chloride anhydrous; about 37.25 mg/l Na₂-EDTA; about 27.8 mg/l ferrous sulfate.7H₂O; about 90.37 mg/l magnesium sulfate; about 3.33 mg/l manganese sulfate H₂O; about 0.8 mg/l potassium iodide; about 2,830 mg/l potassium nitrate; about 400 mg/l potassium phosphate monobasic; and about 1.5 mg/l zinc sulfate.7H₂O.

In addition, the media in the infection step generally excludes AgNO₃. AgNO₃ is generally included in the co-cultivation, resting (when used) and selection steps when N6 media is used. In the co-cultivation step, the immature embryos are co-cultivated with the Agrobacterium on a solid medium. The embryos are positioned axis-down on the solid medium and the medium can include AgNO₃ at a range of about 0.85 to 8.5 mg/l. The embryos are co-cultivated with the Agrobacterium for about 3-10 days.

Following the co-cultivation step, the transformed cells may be subjected to an optional resting step. Where no resting step is used, an extended co-cultivation step may utilized to provide a period of culture time prior to the addition of a selective agent. For the resting step, the transformed cells are transferred to a second medium containing an antibiotic capable of inhibiting the growth of Agrobacterium. This resting phase is performed in the absence of any selective pressures on the plant cells to permit preferential initiation and growth of callus from the transformed cells containing the heterologous nucleic acid. The antibiotic added to inhibit Agrobacterium growth may be any suitable antibiotic; such antibiotics are known in the art and include Cefotaxime, timetin, vancomycin, carbenicillin, and the like. Concentrations of the antibiotic will vary according to what is standard for each antibiotic, and those of ordinary skill in the art will recognize this and be able to optimize the antibiotic concentration for a particular transformation protocol without undue experimentation. The resting phase cultures are preferably allowed to rest in the dark at 28° C. for about 5 to about 8 days. Any of the media known in the art can be utilized for the resting step.

Following the co-cultivation step, or following the resting step, where it is used, the transformed plant cells are exposed to selective pressure to select for those cells that have received and are expressing polypeptide from the heterologous nucleic acid introduced by Agrobacterium. Where the cells are embryos, the embryos are transferred to plates with solid medium that includes both an antibiotic to inhibit growth of the Agrobacterium and a selection agent. The agent used to select for transformants will select for preferential growth of explants containing at least one selectable marker insert positioned within the superbinary vector and delivered by the Agrobacterium. Generally, any of the media known in the art suitable for the culture of sorghum can be used in the selection step, such as media containing N6 salts or MS salts. During selection, the embryos are cultured until callus formation is observed. Typically, calli grown on selection medium are allowed to grow to a size of about 1.5 to about 2 cm in diameter.

After the calli have reached the appropriate size, the calli are cultured on regeneration medium in the dark for several weeks to allow the somatic embryos to mature, generally about 1 to 3 weeks. Preferred regeneration media includes media containing MS salts. The calli are then cultured on rooting medium in a light/dark cycle until shoots and roots develop. Methods for plant regeneration are known in the art (see, e.g., Kamo et al. (1985) Bot. Gaz. 146(3): 327-334; West et al. (1993) Plant Cell 5:1361-1369; and Duncan et al. (1985) Planta 165: 322-332).

Small plantlets are then transferred to tubes containing rooting medium and allowed to grow and develop more roots for approximately another week. The plants are then transplanted to soil mixture in pots in the greenhouse.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claim(s).

That which is claimed:

1. A method for producing food or feed with a reduced amount of phytate, said method comprising:
a) transforming a plant with a nucleic acid molecule comprising a first nucleotide sequence selected from the group consisting of:
i) a nucleotide sequence having at least 90% sequence identity to a nucleotide sequence comprising at least 50 contiguous nucleotides of the nucleotide sequence set forth in SEQ ID NO: 1, 3,4, or 35;
ii) a nucleotide sequence comprising at least 19 contiguous nucleotides of the nucleotide sequence set forth in SEQ ID NO: 1, 3, 4, or 35;
iii) a nucleotide sequence encoding an amino acid sequence that has at least 90% sequence identity to the amino acid sequence set forth in SEQ ID NO:2 or 34; and
iv) a nucleotide sequence which is the complement of i), ii), or iii);
b) growing said plant under conditions in which said nucleotide sequence is expressed; and
c) producing food or feed from said plant,
wherein said plant has a reduced amount of phytate in comparison to a control plant.

2. The method of claim 1, wherein said first nucleotide sequence has at least 95% sequence identity to the nucleotide sequence set forth in nucleotides 90-1226 of SEQ ID NO: 1.

3. The method of claim 1, wherein said plant is further transformed with a nucleic acid molecule comprising a second nucleotide sequence selected from the group consisting of:
a) a nucleotide sequence having at least 90% sequence identity to a nucleotide sequence comprising at least 100 contiguous nucleotides of the nucleotide sequence set forth in SEQ ID NO: 1, 3, 4, or 35;
b) a nucleotide sequence having at least 90% sequence identity to the nucleotide sequence set forth in SEQ ID NO: 1, 3, 4, or 35;
c) a nucleotide sequence comprising at least 50 nucleotides of the sequence set forth in SEQ ID NO: 1, 3, 4, or 35; and
d) a nucleotide sequence which is the complement of (a), (b), or (c).

4. The method of claim 1, wherein said plant is further transformed with a nucleic acid molecule comprising a second nucleotide sequence selected from the group consisting of:
a) an mi1ps nucleotide sequence;
b) an IPPK nucleotide sequence;
c) an ITPK-5 nucleotide sequence;
d) an IP2K nucleotide sequence;
e) an MRP nucleotide sequence;
f) a phytase nucleotide sequence;
g) a nucleotide sequence having at least 90% sequence identity to a nucleotide sequence comprising at least 100 contiguous nucleotides of the nucleotide sequence set forth in SEQ ID NO: 42, 44, 45, 46, or 47;
h) a nucleotide sequence having at least 90% sequence identity to the nucleotide sequence set forth in SEQ ID NO: 42, 44, 45, 46, or 47;
i) a nucleotide sequence comprising at least 50 nucleotides of the sequence set forth in SEQ ID NO: 42, 44, 45, 46, or 47;
j) a nucleotide sequence which is the complement of (a), (b), (c),(d), (e), (g), (h), or (i); and
k) a nucleotide sequence having at least 90% sequence identity to the nucleotide sequence set forth in SEQ ID NO: 48.

5. The method of claim 1, wherein said plant is further transformed with a nucleic acid molecule comprising a second nucleotide sequence conferring a trait of interest.

6. The method of claim 5, wherein said trait of interest is selected from the group consisting of:
a) high oil;
b) increased digestibility;
c) high energy;
d) balanced amino acid;
e) high oleic acid;
f) insect resistance;
g) disease resistance;
h) herbicide resistance;
i) drought tolerance; and
j) male sterility.

7. A transformed plant comprising in its genome at least one stably incorporated nucleic acid molecule having a first nucleotide sequence selected from the group consisting of:
a) a nucleotide sequence having at least 90% sequence identity to a nucleotide sequence comprising at least 50 contiguous nucleotides of the nucleotide sequence set forth in SEQ ID NO: 1, 3, 4, or 35;
b) a nucleotide sequence having at least 90% sequence identity to the nucleotide sequence set forth in SEQ ID NO:1, 3, 4, or 35;
c) a nucleotide sequence comprising at least 19 nucleotides of the sequence set forth in SEQ ID NO:1, 3, 4, or 35; and
d) a nucleotide sequence which is the complement of a), b), or c);
wherein said plant has a reduced level of phytate compared to a control plant.

8. The transformed plant of claim 7, wherein said plant is further transformed with a nucleic acid molecule comprising a second nucleotide sequence selected from the group consisting of:
a) an milps nucleotide sequence;
b) an IPPK nucleotide sequence;
c) an ITPK-5 nucleotide sequence;
d) an IP2K nucleotide sequence;
e) an MRP nucleotide sequence;
f) a phytase nucleotide sequence;
g) a nucleotide sequence having at least 90% sequence identity to a nucleotide sequence comprising at least 100 contiguous nucleotides of the nucleotide sequence set forth in SEQ ID NO: 42, 44, 45, 46, or 47;
h) a nucleotide sequence having at least 90% sequence identity to the nucleotide sequence set forth in SEQ ID NO: 42, 44, 45, 46, or 47;
i) a nucleotide sequence comprising at least 50 nucleotides of the sequence set forth in SEQ ID NO: 42, 44, 45, 46, or 47;
j) a nucleotide sequence which is the complement of (a), (b), (c), (d) (e), (g), (h), or (i); and
k) a nucleotide sequence having at least 90% sequence identity to the nucleotide sequence set forth in SEQ ID NO: 48.

9. The transformed plant of claim 7, wherein said plant is further transformed with a nucleic acid molecule comprising at least one second nucleotide sequence that confers at least one trait of interest on said transformed plant.

10. The transformed plant of claim 9, wherein said trait of interest is selected from the group consisting of:
a) high oil;
b) increased digestibility;
c) high energy;
d) balanced amino acid;
e) high oleic acid;
f) insect resistance;
g) disease resistance;
h) herbicide resistance;
i) drought tolerance; and
j) male sterility.

11. Transformed seed of the plant of claim 7, wherein said seed comprises said first nucleotide sequence.

12. Food or feed comprising the plant of claim 7.

13. Food or feed comprising the transformed seed of claim 11.

14. A method for producing food or feed with a reduced amount of phytate, said method comprising the steps of:
(a) transforming a plant cell with at least one first polynucleotide comprising at least 19 nucleotides of the sequence set forth in SEQ ID NO: 1, 3, 4, or 35;
(b) transforming a plant cell with at least one second polynucleotide having at least 94% sequence identity to the complement of the polynucleotide of step (a);
(c) regenerating a transformed plant from the transformed plant cell of step (a); and
(d) producing food or feed from said transformed plant or from seed of said transformed plant;
wherein said plant has a reduced amount of phytate in comparison to a control plant.


SEQUENCE LISTING (48 sequences)

SEQ ID NO: 1 - DNA, 1632 bp, Zea mays
  5′UTR (18)...(89)
  CDS (90)...(1226)
  3′UTR (1227)...(1581)
gtcgacccacgcgtccgctcgccgccccgagacccccacgcttccctctccgcccccgaactgtggcgcctccccccgccgccgcagcgatgccactcgcggcagagcccgacgacgctcatgaggaaagggagaatcagcagctgctaattacgacgaagggagggcccgggcttgagggactggtggtggggagctactgccacgatgtgctaatccggggcgggcgcatagtgggggagacgctcggcggggctgcggccttcgtgtccaacgtgctcgacgccgcttcgccccagggcgccgcgctcaacgagacatccccctttgtcgttgtggccaaggtgggtcacgacttcatctacgcccgcgcgccggcgtccgcgcggcatccgcctctgctctgctcgtccccaaccacctccttccacgcccagttctcggagaccgccgcctcggcgcacgcccccgaccgggagctccggcgcgtgcgcgcctgcgacccgatctaccccgccgaccttcccgaccgccgcttcgcctacggcctcgctgtcggcgtcgccggggaggtgctaccggagacgctcgagcagatgatcaggctctgccgcacggtgctcgtggacgcgcaggcgctgatccgggcgttcgacggtgacggcgccgtcggtcacgtggcgcttggcgataccccgtacgcgcggcttctgccccgagtggcgttcgttaaggcgtcgtcggaggaggcgccatacgttggggtggaaacgacgaggcggcagtgctgtgtgatcgtcacggaggggagggacggctgccggctgtactgggacggtggggaggcgcacgttgcgccgttccccgccgtacaggtggaccctactggcgccggagacagttttctcgcgggctttgcagccggattgctgtgggggttgtcggcgacggacgccgcgctgctggggaacttctttggcgccgctgctgtatcgcaggtcggcgtgcccaccttccatcccaagatgttgcaggcagttaaagaaatacttgaagagaagacaaggaaacgatctagtccatgtatgaacggcgctagttttaccttggagaagtcaaatatgcacaacgagttacacgcagctctccaagaagctgcggtgctgatgtctgaacagcagcaggctgatccggcgaacggcagtggcggtgatatttgctcggcataggtacctcacagtgaagctgaagcagtcgaacgccaaactgaagtttgtggcaaaataaccagcactgcagtcctgaactcctgatctcacattgagatctgtaaacatggtgccaacaagtggaggaagtttgtacatacgctctctccggcctttacactactattctgctggcaaggccgtcagggatcgtttctaccttgctatcgctgacgaggaaatgaagacaactgaacagttgagctgtggcgcttgcacgcaccatgttttctccgctgaacaagtgcgcatttttgagctttcgggcattcgtgctgttaactttttaccattctatatgtcgacttctaccaaaaaaaaaaaaaaaaaaaaaaaaaaaaaaaaaaaaaaaaagggcggccgc

SEQ ID NO: 2 - protein, 379 aa, Zea mays
MPLAAEPDDAHEERENQQLLITTKGGPGLEGLVVGSYCHDVLIRGGRIVGETLGGAAAFVSNVLDAASPQGAALNETSPFVVVAKVGHDFIYARAPASARHPPLLCSSPTTSFHAQFSETAASAHAPDRELRRVRACDPIYPADLPDRRFAYGLAVGVAGEVLPETLEQMIRLCRTVLVDAQALIRAFDGDGAVGHVALGDTPYARLLPRVAFVKASSEEAPYVGVETTRRQCCVIVTEGRDGCRLYWDGGEAHVAPFPAVQVDPTGAGDSFLAGFAAGLLWGLSATDAALLGNFFGAAAVSQVGVPTFHPKMLQAVKEILEEKTRKRSSPCMNGASFTLEKSNMHNELHAALQEAAVLMSEQQQADPANGSGGDICSA

SEQ ID NO: 3 - DNA, 5005 bp, Zea mays
  promoter (1)...(1379)
  exon (1380)...(2582)
  intron (2583)...(4075)
  exon (4076)...(4622)
  TATA_signal (1077)...(1083)
tgttagactgtgtgtgtgtgagtgtgtgtggcccgacaccactgttgggatgccgacccattagggttagggttgagtctctctatatatgtacccccactttatgcaatatatcaagcacttcactattctacatggtatcaaagggttagggcttcctcccaccccacagccgccacatcctccctgccaaagtccagccgccaccggcttccgcgccgccgccccgggaggctcacccatccctcccggggcgacccttctccccagccggtcgccagtctctggccgccgccacccccaaccggcaggcacgggcccctccggccgcctccatccccttcttcggccagcgtcttcctcctcccccagccggcgagctcgggctcaagcgcgccccgaccagtaggcccgcttctcccccttctccggcgcccgcgcctcctcgactctgctactgctccggccgccggctgctattgctcgatgtcggatggcgccgccggcgccmacgccgaaccccctccctggtgcgcatcccgcccgcgccgcccctgctgccagggccgcccgcgccagattttccgccgccggctgctcctgctccggccaccamctyctyccttcccctccttcggcgccccatgtcgccgcctctcttccccccggtttgacccggcctcggcgctctgcctcgacgcggcccgtgtgcgcccccatccggcacgcccatcactggggccccccatctgccgcgcccgccgccgacgcccccatccgccgcgcccgtcaccggcagcggcgcgatgcttctccccgctcggtggcgagccatgactgtgctcccccagcagctaggcccccagctcccttgtcgtcgtctgcctctccatggcgcgggcatctgccacccctgcggcaacgaatctacgcgcccctccctccggctccggtggcgcaacagcgaccccctcctcgatggctcaaatatccccgacctgactctgccagggccttcctcgccggcgtcccactcgtcggcaatcgcccctcccctcccctacccgctcatgcgctataaaaccatagctaaactattagctagatgtgtttaaattcatttagctaaaaaatagatagtagctaaactattggattattctgatacctctagctaattttagccgctaaattctttctaacatggtagtctagtgacattattgggcctgtgttcaagcgcgatcatcatgattcgtgatgacgaggcagaggtacctctccagccagcctccaggacgacaaggcgacgacctgggatctgacaattccaaattccaacgcgctaagctgccaacgtcgccgcagcggcagcacctcaaacgtacgtcgccgcagcagctcagacgtgcgccgctaccacgtgtcctgccgcacgccccgcgtcagggcatctgtaaagccgcttgtcgccgccccggcgcccaccccgccccccgcgcttttathccccacttcaccgcatctcccccctcgtctacgatgccgttgcgcacctcttytctctcgccgccccgagacccccacgcttccctctccgcccccgaactgtggcgcctccccccgccgccgcagcgatgccactcgcggcagagcccgacgacgctcatgaggaaagggagaatcagcagctgctaattacgacgaagggagggcccgggcttgagggactggtggtggggagctactgccacgatgtgctaatccggggcgggcgcatagtgggggagacgctcggcggggctgcggccttcgtgtccaacgtgctcgacgccgcttcgccccagggcgccgcgctcaacgagacatccccctttgtcgttgtggccaaggtgggtcacgacttcatctacgcccgcgcgccggcgtccgcgcggcatccgcctctgctctgctcgtccccaaccacctccttccacgcccagttctcggagaccgccgcctcggcgcacgcccccgaccgggagctccggcgcgtgcgcgcctgcgacccgatctaccccgccgaccttcccgaccgccgcttcgcctacggcctcgctgtcggcgtcgccggggaggtgctaccggagacgctcgagcagatgatcaggctctgccgcacggtgctcgtggacgcgcaggcgctgatccgggcgttcgacggtgacggcgccgtcggtcacgtggcgcttggcgataccccgtacgcgcggcttctgccccgagtggcgttcgttaaggcgtcgtcggaggaggcgccatacgttggggtggaaacgacgaggcggcagtgctgtgtgatcgtcacggaggggagggacggctgccggctgtactgggacggtggggaggcgcacgttgcgccgttccccgccgtacaggtggaccctactggcgccggagacagttttctcgcgggctttgcagccggattgctgtgggggttgtcggcgacggacgccgcgctgctggggaacttctttggcgccgctgctgtatcgcaggtcggcgtgcccaccttccatcccaagatgttgcaggtttgcaccatttgacttgttcagctccttgtacactcggattgggggagaatcattctgttcatgttgatcaattaagtccttggacattttttttaaatgtgtcacttatcagaggcaaaatcatagctctaacggactgcagtgcaggcaatttccaaatgattgacctatgctggagtagtattctgctgttgggaatttgaggacatatttgtactagttggactaaaaattatctcaaattagccaaggttagctagctagtggtgagaacatgtactatactcctatgcaatcacactcatttggtgtaaacatcactcatttggtgtaaacgtacaatgaaaccatccagagcgttcgatctagatccaactatgtttgtcgttttttgtatttctatacccccacatcaccctttatctattttcttcatgccctatatatttgctaaatataggagggtggatataaaaaacaatatacatcattgatctagattctggatgatttgattgcacgttcgcacccaatatgtgatcgcacatgatatgtcatcttaatttggccaacaactagttgaaaatgtgtctaaacaattagcccacctattagccctcttgtttgaatgcactagagctaatttaagttaatttttagctagccaatgattagctcttgtatccaaacaggccctgaatagatgtgatcttttcaaaatgttgacttccgacgatattatcaagggctcaaccatctggcaaccatttattagggtgtctttgagactgaaggaaatttagcagccataaaagggcaggctggtgggcaaaaaaaaaaagagccttgggtggaactatggggtattagttagaaatcaaccagttaataaaccctaaaggctaaaaatgagaacaatgagtatatgtgtctctggtgaagtttccactctagtatgatccttctacttacggtgtgtgcctgcaaaatatgttttgtggctagcatcagcataatatcttgacctatcacttctcattcaatctatgtagatggttgctttgtgactaggttggttcgaagaacatattctaacatccacgatattgccatccctcttttttctgagaaccgctgatacagttattaattggcatactcacaattttttttgccacattcaggacccccctcaagatcattttagggcggccaatgtagtagccttagaatctgttaccagtttccgaggttaacagtttttcatttttgatccagtttccaggggtaaaaatcttgattttttgtcagaataaatagtttcatatcctttgctgctcgacagttgagcattgtacttgcatatagaatttcattttgttaggggtatacctcaacatcgttggcttgttatctggtgtgcttttaatgttttgttcctatggaatcaatgatcttttttcttagttcaagttttcgactgggtgcaggcagttaaagaaatacttgaagagaagacaaggaaacgatctagtccatgtatgaacggcgctagttttaccttggagaagtcaaatatgcacaacgagttacacgcagctctccaagaagctgcggtgctgatgtctgaacagcagcaggctgatccggcgaacggcagtggcggtgatatttgctcggcataggtacctcacagtgaagctgaagcagtcgaacgccaaactgaagtttgtggcaaaataaccagcactgcagtcctgaactcctgatctcacattgagatctgtaaacatggtgccaacaagtggaggaagtttgtacatacgctctctccggcctttacactactattctgctggcaaggccgtcagggatcgtttctaccttgctatcgctgacgaggaaatgaagacaactgaacagttgagctgtggcgcttgcacgcaccatgttttctccgctgaacaagtgcgcatttttgagctttcgggcattcgtgctgttaactttttaccattctatatgtcgacttctaccaaaaggtctagcgtttttaccctgactgaacacagggaaatttgtctgactgaactgagaagggccaacacacaagttagcgcgtcaccgatgtctgaattagagttggacttccggtcactggaaaacagaaccaatagaaataacagaaattattcaacaatctggtgccatgataagctctacacatatacacaatccagattcaaaatcactgatcagttaaaaaacgattcacaacagcaggtggatcatcttggattcggcgtttaaacttaataaacaatgctacatccgtaacaaaaggaactcaagtaattgtggctgacacaaagtcggccatataaaatttgaagttctccgtactttggagataggcatcc

SEQ ID NO: 4 - DNA, 1379 bp, Zea mays
  promoter (1)...(1379)
  TATA_signal (1077)...(1083)
tgttagactgtgtgtgtgtgagtgtgtgtggcccgacaccactgttgggatgccgacccattagggttagggttgagtctctctatatatgtacccccactttatgcaatatatcaagcacttcactattctacatggtatcaaagggttagggcttcctcccaccccacagccgccacatcctccctgccaaagtccagccgccaccggcttccgcgccgccgccccgggaggctcacccatccctcccggggcgacccttctccccagccggtcgccagtctctggccgccgccacccccaaccggcaggcacgggcccctccggccgcctccatccccttcttcggccagcgtcttcctcctcccccagccggcgagctcgggctcaagcgcgccccgaccagtaggcccgcttctcccccttctccggcgcccgcgcctcctcgactctgctactgctccggccgccggctgctattgctcgatgtcggatggcgccgccggcgccmacgccgaaccccctccctggtgcgcatcccgcccgcgccgcccctgctgccagggccgcccgcgccagattttccgccgccggctgctcctgctccggccaccamctyctyccttcccctccttcggcgccccatgtcgccgcctctcttccccccggtttgacccggcctcggcgctctgcctcgacgcggcccgtgtgcgcccccatccggcacgcccatcactggggccccccatctgccgcgcccgccgccgacgcccccatccgccgcgcccgtcaccggcagcggcgcgatgcttctccccgctcggtggcgagccatgactgtgctcccccagcagctaggcccccagctcccttgtcgtcgtctgcctctccatggcgcgggcatctgccacccctgcggcaacgaatctacgcgcccctccctccggctccggtggcgcaacagcgaccccctcctcgatggctcaaatatccccgacctgactctgccagggccttcctcgccggcgtcccactcgtcggcaatcgcccctcccctcccctacccgctcatgcgctataaaaccatagctaaactattagctagatgtgtttaaattcatttagctaaaaaatagatagtagctaaactattggattattctgatacctctagctaattttagccgctaaattctttctaacatggtagtctagtgacattattgggcctgtgttcaagcgcgatcatcatgattcgtgatgacgaggcagaggtacctctccagccagcctccaggacgacaaggcgacgacctgggatctgacaattccaaattccaacgcgctaagctgccaacgtcgccgcagcggcagcacct

SEQ ID NO: 5 - skipped

SEQ ID NO: 6 - protein, 379 aa, Zea mays
MPLAAEPDDAHEERENQQLLITTKGGPGLEGLVVGSYCHDVLIRGGRIVGETLGGAAAFVSNVLDAASPQDAALNETSPFVVVAKVGHDFIYARAPASARHPPLLCSSPTTSFHAQFSETAASAHAPDRELRRVRACDPIYPADLPDRRFAYGLAVGVAGEVLPETLEQMIRLCRTVLVDAQALIRAFDGDGAVGHVALDDTPYARLLPRVAFVKASSEEAPYVGVETTRRQCCVIVTEGRDGCRLYWDGGEAHVAPFPAVQVDPTGAGDSFLAGFAAGLLWGLSATDAALLGNFFGAAAVSQVGVPTFHPKMLQAVKEILEEKTRKRSSPCMNGASFTLEKSNMHNELHAALQEAAVLMSEQQQADPANGSGGDICSA

SEQ ID NO: 7 - protein, 323 aa, Artificial Sequence
  Pfam consensus for pfkB family
KVVVIGEANIDLIPTVEGLPEGELNERVKSFEKGPGGAGANVAVALARLGNPSYGKVAFIGKVGDDEFGEFLLELLKKEGVDTDYVKVDEGTRTGLALVLVDGDGGERTIVFYRGANADLTLEELDEDLLEEADILHLSGISLVLLPEPLPEETLEALAEAAKAGGKISFDPNLRDPLWSDEEALEVLLELLPLADILKPNEEELELLTGLKGEVEEALAALHKLLAKGAVTKLVVVTLGADGALLVTKGGEVHVPAVPKVKVVDTTGAGDAFVAGFLAGLLATDDSNTQLDGKDLEEALRFANAAAALVVQKKGAISSLPTR

SEQ ID NO: 8 - DNA, 8 bp, Artificial Sequence
  primer
accagccc

SEQ ID NO: 9 - DNA, 48 bp, Artificial Sequence
  primer
gtaatacgactcactatagggcacgcgtggtcgacggcccgggctggt

SEQ ID NO: 10 - DNA, 32 bp, Artificial Sequence
  primer
agagaagccaacgccawcgcctcyatttcgtc

SEQ ID NO: 11 - DNA, 22 bp, Artificial Sequence
  primer
gtaatacgactcactatagggc

SEQ ID NO: 12 - DNA, 29 bp, Artificial Sequence
  primer
ctcttcgtcyataatggcaattatctcaa

SEQ ID NO: 13 - DNA, 29 bp, Artificial Sequence
  primer
ctcttcgtcyataatggcaattatctcat

SEQ ID NO: 14 - DNA, 29 bp, Artificial Sequence
  primer
ctcttcgtcyataatggcaattatctcac

SEQ ID NO: 15 - DNA, 29 bp, Artificial Sequence
  primer
ctcttcgtcyataatggcaattatctcag

SEQ ID NO: 16 - DNA, 29 bp, Artificial Sequence
  primer
ctcttcgtcyataatggcaattatctcta

SEQ ID NO: 17 - DNA, 29 bp, Artificial Sequence
  primer
ctcttcgtcyataatggcaattatctctt

SEQ ID NO: 18 - DNA, 29 bp, Artificial Sequence
  primer
ctcttcgtcyataatggcaattatctctc

SEQ ID NO: 19 - DNA, 29 bp, Artificial Sequence
  primer
ctcttcgtcyataatggcaattatctctg

SEQ ID NO: 20 - DNA, 29 bp, Artificial Sequence
  primer
ctcttcgtcyataatggcaattatctcca

SEQ ID NO: 21 - DNA, 29 bp, Artificial Sequence
  primer
ctcttcgtcyataatggcaattatctcct

SEQ ID NO: 22 - DNA, 29 bp, Artificial Sequence
  primer
ctcttcgtcyataatggcaattatctccc

SEQ ID NO: 23 - DNA, 29 bp, Artificial Sequence
  primer
ctcttcgtcyataatggcaattatctccg

SEQ ID NO: 24 - DNA, 29 bp, Artificial Sequence
  primer
ctcttcgtcyataatggcaattatctcga

SEQ ID NO: 25 - DNA, 29 bp, Artificial Sequence
  primer
ctcttcgtcyataatggcaattatctcgt

SEQ ID NO: 26 - DNA, 29 bp, Artificial Sequence
  primer
ctcttcgtcyataatggcaattatctcgc

SEQ ID NO: 27 - DNA, 29 bp, Artificial Sequence
  primer
ctcttcgtcyataatggcaattatctcgg

SEQ ID NO: 28 - protein, 397 aa, Oryza sativa
MAPSPAAAMPLAAEPDEVVVEVEEEEERGVKGGGGVAGLDEVEGLVVGSYCHDVLLRGGRVVGETLGGAAAFVSNVLDAASPAGASLAVVSKVGHDFAYATAAAPARHPPVLCASPTTSFHARFSDDAASAHAPDRQLRRVHACDPIYPADLPDRRFAYGLAVGVAGEVLPETLERMIRLCRAVLVDAQALIRAFDGEAKGGGAVRHVALEATPYARLLPRVAFLKASSEEAPYVGVETARRRCCVIVTEGRDGCRLYWDGGEARVAPFPAVQVDPTGAGDSFLAGFASGLLWGLSATDAALLGNFFGAAAVSQVGVPTFDPKMLQAVKQILEKAVKRPCTHINGNTFTFQRSSMHDELHKSLQEAAMLVCEQKQANSPATDNGDVCSINELTSLPS

SEQ ID NO: 29 - protein, 353 aa, Arabidopsis thaliana
MTLNPPPSQQTSHSPRRIPNRRVLIVGNYCHDVLIQNGSVVAETLGGAASFISNVLDSSSVSCDLVSKVGHDFRYEVTHSPIVAPEKETTVFEAYFDLGIDGIGHADRVLKRVSACDPILPSDIPDSRFDFGMAVGVGGEILPETLEKMVEICDVVAVDIQALIRVFDPVDGAVKLVDLKESGFYHILHRIGFLKASSDEALFMDVEQMKHLCCVVVTNGEKGCRIYHKDDEMTVPPFLAKQVDPTGAGDSFLGGLIVGLVEGLTVPDAALLGNLFGSITVEHIGQPKFDLMMLQKVKDEVQRRKKQCNISSSHNNDHNEFHERLSPARFSCVDSQLQPKLLVNGHSCDDRSL

SEQ ID NO: 30 - protein, 380 aa, Sorghum
MLLAAEPDDAHEEGENQQLLITTKGGPGLEGLVVGSYCHDVLIRGGRVVGETLGGAAAFVSNVLDAASPPGRCAQRDSPPSSSWPRWAHDFVYASAPAPARHPPLLCASPTTSFHAQFSETAASAHAPDRELPRVRACDPIYPADLPDRRFPTALAVGVAGEVLPETLERMIRLCRAVLVDAQALIRAFDGDGAVGHVALDDTPYARLLPRVAFVKASSEEAPYVGVETTRQRCCVIVTEGRDGCRLYWDGGEARVAPFPAVQVDPTGAGDSFLAGFATGLLWGLSATDAALLGNFFGAAAVSQVGVPTFHPKMLQAVKEILEEKTTKRYSPCINGTSFTFEKSNLHNELHAALQEAAMLMSEQQQADKANGNGGDICSP

SEQ ID NO: 31 - protein, 320 aa, Brassica oleracea
MTLTPPPPQQTPNSPPRVLIVGNYCHDVLIQNGTVVAETLGGAASFISNVLDSSSVSCDLISKVGRDFKYDVAHPPIVAPEKETTVFEAYFDLAADRVLKRVAACDPILPSDIPDSRFDIGMAVGVGGEILPETLEKMVEICDVVAVDIQALIRVFDPVDGAVKLVDLKESGFRHLMGKIGFLKASSDEALFMDVEGMRRLCCVVVTGEKGCRIYDKDDETVVPPFVAKQVDPTGAGDSFLGGLVVGLVEGLDVPDAALLGNLFGSITVEHIGQPKFDSMMLQVLFLYLSGFCLKVVELHAVRSVACCYHHHEHRTSSGV

SEQ ID NO: 32 - protein, 78 aa, Helianthus annuus
PQNSPPSTPLTMVANQPPHGLIVGNYCHDVLIKNNAVLAETLGGAAAFISAVLDGLHLPFTYVTKAGSDFAYTTTVNH

SEQ ID NO: 33 - protein, 128 aa, Helianthus annuus
TVYWKDGEVRIPAFSSVQVDPTGAGDSFLGGLVAGLVEGLAVPDAAVLGNFFGSLTVEQIGLPEFDVRLMQKVKDEVHARKMQRDGHDDESRFTKTSGHEQFIASLVAAKLIKTQTVSPNHVLDPDMR

SEQ ID NO: 34 - protein, 375 aa, Glycine max
MVTDSKPIIPRRGLVVGNYCHDVLHRDGETVAETLGGAASFISVILDALSLPFHTVSKVGPNFAYTADTSNHPPLIIPTSQTTLFHAHFGSGHPDRLLNRVRSCDSIQPTDLPDQQPRFGFGLAVGVGGEILPETVEKMLSICDHVFVDIQALIRRFDNINGRVSHVALRESGFFHLLPRVAFLKASADEAVFIDVEEVRKWCCVVVTHGKDGCEVFCENGAFKVAPFEADQVDPTGAGDCFLGGFAAGIVRGLAVPDAALLGNFFGSIAVAQVGPPKLDFNLIQMVKDEMHKRKLQDIPFLERRDESPGFQKPPEQDQFYASLVTAKAIITCQIQESGRNLLSSPKVFEQNNAKTRLSLASVHEEPIPSVDGKP

SEQ ID NO: 35 - DNA, 1511 bp, Glycine max
ctcattctggcgcgcgtcctcacatccccacagttaccactattcctcttttcttcatcatcatcttcttcttcttcttcttcttcttcttgcatttacatcaactcttctcccaacccataaaaaaaaaaaaaaagttcgtattttccctcccatgatcgaccctcatggtcacagactccaagcccatcattccccggcgcggcctcgtcgtcggcaactactgtcatgacgtcctccaccgcgacggcgagaccgtcgccgagaccctcggcggcgccgcctccttcatctccgttatcctcgatgccctctcccttcccttccacaccgtctccaaggtgggccccaacttcgcctacaccgccgacacatcgaatcaccccccgttaataatcccaacctcccaaaccacgctcttccacgctcacttcggttcgggccacccggaccgcctcctgaaccgcgtccgatcctgcgattcaatccaacccaccgacctcccggaccaacaaccccggttcgggttcggactcgcggtcggggtcggcggtgagattctcccggagacagtggaaaaaatgctctcgatctgcgaccacgtgttcgtggacattcaggctctgattcgcaggttcgacaacatcaacggtcgcgtgagccacgtggcgctaagagagagcgggttcttccacctcttgccgcgtgtggcgtttctcaaggcttctgcggatgaggccgtttttattgacgtggaggaggtgaggaagtggtgttgcgtggtcgtcacgcacgggaaggatggttgcgaggtgttttgtgaaaacggtgcgtttaaggtggcgccgttcgaggctgatcaggttgatcccaccggcgccggggactgttttctcggcgggtttgcggcggggattgtgaggggcctggccgtgcctgatgcggctctgttggggaacttctttggatccattgctgtggcgcaagtgggaccgcccaagcttgacttcaacttgattcagatggttaaggatgagatgcacaaaaggaagctgcaggatattcccttcttagaaagaagagatgagtcgccagggtttcagaagccacctgaacaagatcaattctatgcatctcttgttactgccaaagccataattacttgccagattcaagaatctggtcggaatctgctaagctctcctaaagtgtttgagcaaaataatgcaaagacaagactttcactggcttctgtgcacgaggaaccaattccaagcgttgatggtaaaccctaacatgggatggttggagttccaaatctcactgtaactctccagtgcccttccttttcatatcaaaaatagagatttcttagcatccaggaatctgtcaatttatggtgccttttccacacctagtctcacgtgcagcctaatttgtagttctacttgtaccaaattactaataaattattcttaaaaagaaaatcgagattgaaattccattcaaaa

SEQ ID NO: 36 - protein, 34 aa, Artificial Sequence
  Conserved Domain A
  VARIANT (2)...(2): Can be Val or Ile
  VARIANT (12)...(12): Can be Ile or Leu
  VARIANT (13)...(13): Can be Arg or Lys
  VARIANT (17)...(17): Can be Val or Ile
  VARIANT (18)...(18): Can be Val or Leu
  VARIANT (19)...(19): Can be Ala or Gly
  VARIANT (27)...(27): Can be Ala or Ser
  VARIANT (29)...(29): Can be Ile or Val
  VARIANT (32)...(32): Can be Val or Ile
  VARIANT (0)...(0): Xaa at other positions can be any amino acid
LXVGXYCHDVLXXXGXXXXETLGGAAXFXSXXLD

SEQ ID NO: 37 - protein, 61 aa, Artificial Sequence
  Conserved Domain B
  VARIANT (15)...(15): Can be Ala or Ser
  VARIANT (25)...(25): Can be Gly or Ala
  VARIANT (26)...(26): Can be Leu or Met
  VARIANT (31)...(31): Can be Ala or Gly
  VARIANT (34)...(34): Can be Val or Ile
  VARIANT (43)...(43): Can be Val or Ile
  VARIANT (45)...(45): Can be Leu or Ile
  VARIANT (0)...(0): Xaa at other positions can be any amino acid
RXLXRVXACDPIXPXDLPDXRFXXXXAVGVXGEXLPETLEXMXXXCXXVXVDXQALIRXFD

SEQ ID NO: 38 - protein, 37 aa, Artificial Sequence
  Conserved Domain C
  VARIANT (13)...(13): Can be Gly or Ala
  VARIANT (19)...(19): Can be Leu or Ile
  VARIANT (20)...(20): Can be Val or Leu
  VARIANT (30)...(30): Can be Leu or Val
  VARIANT (37)...(37): Can be Ser or Ala
  VARIANT (0)...(0): Xaa at other positions can be any amino acid
QVDPTGAGDSFLXGXXXGXXXGLXXXDAAXLGNFFGX

SEQ ID NO: 39 - protein, 20 aa, Artificial Sequence
  Conserved Domain
  VARIANT (13)...(13): Can be Gly or Ala
  VARIANT (19)...(19): Can be Leu or Ile
  VARIANT (20)...(20): Can be Val or Leu
  VARIANT (0)...(0): Xaa at other positions can be any amino acid
QVDPTGAGDSFLGGXXXGLV

SEQ ID NO: 40 - protein, 363 aa, Artificial Sequence
  Consensus sequence
  VARIANT (0)...(0): Xaa can be any amino acid
PXXXXXXXXXXXXXXXXXXXXXXXXXXXXGLVVGXYCHDVLIRXGXVVAETLGGAAAFISNVLDAASXXXXXXXXXXXXXXXVSKVGXDFXYXXXXAXXXXPXXXXXXXTTXFXAXFXXXAXXXXXXXRXLXRVXACDPIXPADLPDXRFXXGLAVGVAGEVLPETLEXMIXLCXXVXVDXQALIRXFDXXXXXXXAVXXVXLXXTXYXXLLXRVAFLKASSDEAXFVXVEXXRXXCCVIVTXGXXGCRLYXXXGEXXVXPFXAXQVDPTGAGDSFLGGXXXGLVXGLSXXDAALLGNFFGSXXVQVGXPXFDXXMLQXVKDXLXXRXXXXXXXXXXXXXXXXXXXXXXXEXXXAXXXXAXXI

SEQ ID NO: 41 - protein, 388 aa, Artificial Sequence
  Consensus sequence
  VARIANT (0)...(0): Xaa can be any amino acid
MPLAAEPDDAHEEXENQQLLITTKGGXXXXXPGLEGLVVGSYCHDVLIRGGRVVGETLGGAAAFVSNVLDAASPXGXXAXXXXXPXXVVSKVGHDFXYAXAPAPARHPPLLCASPTTSFHAQFSETAASAHAPDRELRRVRACDPIYPADLPDRRFAYGLAVGVAGEVLPETLERMIRLCRAVLVDAQALIRAFDGDGXXXXAVGHVALXDTPYARLLPRVAFXKASSEEAPYVGVETTRRRCCVIVTEGRDGCRLYWDGGEARVAPFPAVQVDPTGAGDSFLAGFAXGLLWGLSATDAALLGNFFGAAAVSQVGVPTFXPKMLQAVKEILEEKTXKRXSPCINGNSFTFEKSNXHNELHAALQEAAMLMSEQQQADXANGSGGDICS

SEQ ID NO: 42 - DNA, 1961 bp, Zea mays
gtcgacccacgcgtccggcccaacaaaggagcgcggcggcccctccttccttcctcccacttctctcgcgcggcgctcgcttacctcgcctcgcattccgttcgagcaggggagcggcagtgagaagggagggaattaaggcaagatgttcatcgagagcttccgcgtcgagagcccccacgtgcggtacggcccgacggagatcgagtcggagtaccggtacgacacgacggagctggtgcacgaggccaaggacggcgcctcccgctgggtcgtccgccccaagtccgtcaagtacaacttccggaccagcaccgcggtccccaagctcggggtcatgcttgtggggtggggaggcaacaacgggtccacgctgacggctggggtcattgccaacagggaggggatctcatgggcgaccaaggacaaggtgcagcaagccaactactacggctccctcacccaggcttccaccatcagagtaggcagctacaacggggaggagatatatgcgccgttcaagagcctcctacccatggtgaacccagacgaccttgtgtttggaggctgggacatcagcagcatgaacctggcagatgccatgaccagggccaaggtgctggacattgacctgcagaagcagctcaggccctacatggagtccatggtgccacttcccggtgtctatgatccggacttcatcgccgctaaccagggctctcgtgccaacaatgtcatcaagggcaccaagaaagaacaggtggagcagatcatcaaagatatcagggagtttaaggagaagaacaaagtggacaaggtagttgtgctgtggactgcaaacactgaaaggtacagcaatgtatgtgctggtctcaacgacacaatggagaatctgctggcatctgtggacaagaacgaggcggagatctcgccatcaacactatatgccattgcctgtgtcacggagggggtgccgttcatcaatgggagcccccagaacacttttgtgcctgggctgattgatcttgctatcaagaacaactgcctgatcggtggtgacgacttcaagagtgggcagaccaagatgaaatcggtcctggttgattttcttgttggtgctggaataaagcccacctcgattgtgagctacaaccacttgggaaacaacgacggcatgaacctgtctgcccctcaaacattcaggtccaaggagatctccaagagcaacgtggtggatgacatggtctcaagcaatgccattctctatgggcccggcgagcatcccgatcatgttgttgtcatcaagtatgtgccgtatgtgggagacagtaagagggctatggacgagtacacatcagagatcttcatgggcggcaagagcaccatcgtgctgcacaacacctgcgaggactcgctcctcgccgcaccgatcatcctcgatctggtgctcctggctgagctcagcaccaggatccagttaaaacctgagggaacggacaagttccactccttccacccggtggccaccatccttagctacctcaccaaggcaccactggttccacccggcacaccggtggtgaacgctcttgcaaagcagagggcgatgctggagaacatcatgagggcttgcgttggcctggccccagagaacaacatgatcctggagtacaagtgagcgaagtggcgtggcctgcagctagatatggaggaggctgcacgaaggggactagagaggcgagattagctgtggaattgtgttggcttctcgtgttttcttttgcgttcttttcctggtcatcgctgtggcgcttttgtattttatttgttggacccgtaacactatcagggctctgctattagcgcttgaagcctgtaatggcattggcatcgtatgataatgtgatcgagggtgctagttcccctaaaaaaaaaaaaaaaaaaaagggcggccgctctag

SEQ ID NO: 43 - DNA, 19 bp, Artificial Sequence
  primer
actatagggcacgcgtggt

SEQ ID NO: 44 - DNA, 1170 bp, Zea mays
gaaaatctctttctccgctgcgctgcaaacccaccgcttccaccatcgccactcgtcaccccttgctcccatagtccccataccatgcccgacctccacccgccggagcaccaagtcgccggtcaccgcgcctccgccagcaagctgggcccgctcatcgacggctccggcctcttctacaagccgctccaggccggcgaccgtggggagcacgaggtcgccttctatgaggcgttctccgcccacgccgccgtcccggcccgcatccgagacaccttcttcccccggttccacggcacgcgactcctccccaccgaggcgcagcccggggagccgcatccgcacctcgtcctcgacgacctcctcgcggggtttgaggcgccctgcgtcgcagacatcaagatcggcgccatcacgtggccaccgagttcgccggagccctacatcgccaagtacctcgccaaggaccgcgggaccacgagcgttctgctcggattccgcgtcttgcgtccgagtcgtcggccccgagggcgccgtgtggcggacggagcgcccggaggtgaaggctatggacaccgtcggcgtccgccgcgtgctccggcgctacgtgtcatccgcttgccgacgaggggatggactgcgcgctcgcggcggcggtgtacggaggaaaaggtggagtcttgtcacagctgcgcgagctcaaggcatggttggaggagcagactctgttccacttctactcggcgtcgattcttctgggctatgatgctgctgcagtcgcagcaggcggaggtgggggtggggtaacagtgaagctggtggactttgcccatgtgcccgagggtgatggggtgattgaccacaacttcctgggcgagctctgctagctgatcaagttcgtttctgacattgttccagagactccttagacgcagcctttgggtccttcttaagagaggatcctgacatttttgatttgataacaaaggaagcactttcagctgcaaaaaaagaaagcagcagtgaggatgaagatgacagtagtgaggaaagttcggatgatgagccaacaaaagttgaagaaaagaaggctccaaaagtatcagaaaacattggatctgaggatgaatcttctgaagacgagagtgataaagacagtgaagagcctca

SEQ ID NO: 45 - DNA, 1388 bp, Zea mays
ccacgcgtccgcaaatttcaatctccatcgatcgattcctcccgaacccgacccgatggcctccgacgccgccgccgagccctcctccggcgtcacccaccccccgcgctacgtcatcggttacgcgctcgcgccgaagaagcagcaaagcttcatccagccgtcgctggtggcccaggcggcgtcgcggggcatggacctcgtccccgtggatgcgtcgcagcccctggcagagcaagggcccttccacctcctcatccacaagctctacggagacgactggcgcgcccagctcgtggccttcgccgcgcgccacccggccgtccccatcgtcgacccgccccacgccatcgaccgcctccacaaccgcatctccatgctccaggtcgtctccgagctcgaccacgccgccgaccaggacagcactttcggtatccccagccaggtcgtcgtctacgacgctgccgcgctcgccgacttcggactccttgccgcgctccgcttcccgctcatcgccaagcccctcgtcgccgacggcaccgccaagtcccacaagatgtcgctcgtctaccaccgcgagggcctcggcaagctccgcccgccgcttgtgctccaggagttcgtcaaccatggcggcgtcatcttcaaggtctacgtcgtcggcggccacgtcacttgcgtcaagcgccgtagcctgcccgacgtgtcccccgaggatgacgcatcggcccagggatccgtctccttctcccaggtctccaacctccccactgagcgcacggcggaggagtactacggcgaaaagagtctcgaggacgccgtcgtgccgcccgccgcattcatcaaccagatcgcgggcggcctccgccgcgcgctgggcctgcaactcttcaacttcgacatgatccgcgacgtccgcgccggcgaccgctatctcgtcattgacatcaactacttcccgggctacgccaagatgccaggatacgagactgtcctcacggatttcttctgggagatggtccataaggacggcgtgggcaaccaacaggaggagaaaggggccaaccatgttgtcgtgaaataagatgatgattgatggcactggatatctggcgaatgctgctgattctggatgcagaattcgatgaggggatttagttggttgtagtatctggcgaatgctgctggttctggatgcagaatttgatgaggggatttagttggatttcaacccatagcatgccgaggacctcctagctctttccaaaccagttgtttaggtatcttttctgggtaagtcagcttcatctagtttagtctgtctgaacaaaagagtgggacatgacccaaacggaattctaatgaaaaacgagctctctatctgcaaa

SEQ ID NO: 46 - DNA, 1932 bp, Zea mays
gtcgacccacgcgtccgagcaccagcatctcttcaggtctccaccaagcgcagacaccgcagcagcggcagcggcacgatctggtgacccccccgccgcgtcaagcctgctcctccggtgatcgccggactggcggggtaggaaccagcggagcgcagcccgcctccttccgctgtgtctgacagcagcagatcctcgatggagatggatggggttctgcaagccgcggatgccaaggattgggtttacaagggggaaggcgccgcgaatcttatcctcagctacaccggctcgtcgccctccatgcttggcaaggtactgcggctcaagaagattctaaaaaacaagtcgcagcgggcaccaagttgtattgtattctcaagtcatgagcaactcctgtggggccatatcccagaactggttgagtcggtcaaacaagattgcttggctcaagcctatgcagtgcatgttatgagccaacacctgggtgccaatcatgtcgatggtggggtccgtgtacgtgtttctagggattttctggagcttgtcgaaaagaatgttcttagcagccgtcctgctgggagagtaaatgcaagttcaattgataacactgctgatgccgctcttctgatagcagaccactctttattttctggcaatcctaagggtagcagctgcatagctgtagagataaaggccaaatgtgggtttctgccatcatcagaatatatatcagaagataatactatcaagaaacaagtaacgagatataagatgcatcagcacctcaaattttatcagggtgagatatcgaagactagtgagtacaatcctcttgatctattttctgggtcaaaagagagaatatgcatggccatcaagtcccttttctcaactcctcagaacaacttaaggatttttgtcaatggatctttagcttttggtggcatgggaggtggtgcagatagtgttcatcctgctgacactcttaagtgtcttgaagatctcagcaagattagtggcctaaaactccctgacttcactgagctcctgtcagagacaatttttaggtctgaggtattaggcaacctgttggccactcaaaagttggatgatcatgacattgaaggggtaattcatctgtactacaacataatttctcagccttgtttagtctgcaaaaacctaactgatgtagagctattgcggaagtacactttcttgcattctcttccgttggacaaaagcctgaagatcgttagggacttcctcatttctgctaccgcaaaggactgtagcctgatgatcagctttcggccaagagagaatggtagtacagattctgagtatgattcagtgtttcttgaatcagtgaagcgaacctatgagtacaaggcatatttccttgatctggatgtgaaacctctggataagatggagcattattttaaactggatcagaggatagtcaatttctacacaagaaatgggggaggtcttgccatctccaaagggcagtaataccaaagacacttcgaggatttatacatctggagaagggtgcatcagggagtgttggttgttgttcctgctgcttggtgctgctgttgtaacttcatgagtacagtcccaaggttgggaggctcgacccttaacgcctggaaagggcacagggagctgtgttgtccgtcagtcgctgttgtaactcaaactagtgcatacaccgtggcttgtcacggtaatttccgaagatgtccaacgttagttgagacaaccgaactgcttaccgtggcaatcactcattgtaacatcaagttgaaaatgagggctgaagtttccctcacaggctaccatatgtcagatatgtcctttgtaccactaataagtgcccctggggtcatgtatgaatgtatctcaatttgctattgcaaa

SEQ ID NO: 47 - DNA, 5139 bp, Zea mays
cctctctctcccctcctcgaaccaggcgcaggcgagcgtctctgcccgcccgcctgctgctaccgccaaaacgcctcctttgttgccatccgccgatgccgtaatccgccgcccaaagctcttcctttttccctctctctcgcccgcggccgcactccctgccccagtgcctgccgtggcgagcccaaccccaatgccttttaaacccctccccgctccctcactgatccccaccgcctcccaatgccgccctccttcccctccctcccgctcccggaggccgttgccgccaccgcccacgccgcgctgctcgcgctcgccgcactcctgctcctcctccgcgccgcgcgcgcgctcgcctcccgctgcgcgtcatgcctcaaggcgccgcgccgccgcgggggccccgccgtcgtcgtgggcgacggcgccggcggcgccctcgcggcggcgactgccggcgcctggcacagggccgtgctggcgtcctgcgcctacgccctgctctcgcaggtcgccgtgctgagctacgaggtggccgtcgccggctcgcgcgtctcggcgcgggcgctgctgctgccggccgtgcaggcggtgtcctgggccgcgctgctggcgctcgcgcttcaggcccgcgccgtcggctgggccaggttccctgcgctggtgcggctctggtgggtggtctccttcgcgctctgcgttgtcattgcgtacgacgactccaggcgcctgataggccagggcgcgcgcgctgtggattacgcgcacatggttgccaacttcgcgtccgtgccggccctgggcttcctgtgcttggttggtgtcatgggttccaccggtttggaattggagtttacggaggatggcaacggcctgcatgagccgctgctgctcggcaggcagcgcagagaggcagaggaggagctcggctgtctgagggtcactccctacgctgatgctgggatcctcagccttgcaacattgtcatggcttagtccgttgctctctgttggtgcgcagcggccacttgagttggctgacatacccttgctggcgcacaaggaccgtgcaaagtcatgctataaggcgatgagcgctcactacgagcgccagcggctagaataccctggcagggagccatcactcacatgggcaatactcaagtcattctggcgagaggccgcggtcaatggcacatttgctgctgtcaacacgattgtgtcgtatgttggaccttacttgatcagctattttgtggactacctcagtggcaacattgctttcccccatgaaggttacatccttgcctctatattttttgtagcaaaactgcttgagacactcactgcccgacagtggtacttgggtgtggacatcatggggatccatgtcaagtctggcctcactgccatggtgtataggaagggtctccgactgtcaaacgcctcacggcagagccacacgagtggtgagattgtgaattacatggccgtcgatgtgcagcgtgtgggggactatgcatggtatttccatgacatctggatgcttcccctgcagatcattcttgctctcgccatcctgtacaagaacgtcgggatcgccatggtttcaacattggtagcaactgtgctatcgatcgcagcctctgttcctgtggcaaagctgcaggagcactaccaagataagttaatggcatcaaaagatgagcgcatgcgcaagacttcagagtgcttgaaaaatatgaggattttgaagcttcaggcatgggaggatcggtaccggctgcagttggaagagatgaggaacgtggaatgcagatggcttcggtgggctctgtactcacaggctgcagttacatttgttttctggagctcgccaatctttgtcgcagtcataacttttgggacttgcatattactcggtggccagctcactgcaggaggggttctatccgctttagcaacgtttcggatcctccaagagcctctgaggaacttcccggatctcatctctatgatggcacagacaagggtgtctttggaccgtttgtctcattttctgcagcaagaagaactgccagatgacgcaactataaatgttccacaaagtagtacagataaggcagtcgatattaaggatggcgcattctcttggaacccatacactctgacccctacactttctgatatacaccttagtgtagtgagaggcatgagagtagcagtctgtggtgtcattggttctggtaaatcaagtctactatcgtctatactcggggagatacccaaattatgtggccatgtcaggataagtggcacagcagcgtatgttcctcagactgcatggatacagtctggaaatattgaggagaatattctgtttggcagtcaaatggatagacaacgttacaagagagtcattgcagcttgctgtcttaagaaagatcttgagctgctccagtacggagatcagactgttattggtgatagaggcattaatttgagtggaggtcagaaacaaagagttcagcttgctagagcactctaccaagatgctgatatttatttgcttgatgatcccttcagtgctgttgatgctcatactgggagcgaactgtttaaggagtatatattgactgcactagcaaccaaaacagtaatctatgtaacacatcaagttgaatttctaccagctgctgatctgatattggttcttaaggatggccatatcacacaagctggaaagtatgatgatcttctgcaagctggaactgatttcaatgctctggtttctgctcataaggaagctattgaaaccatggatatatttgaagattccgatagtgatacagtttcttctattcccaacaaaagattgacaccaagtatcagcaatattgataacctgaaaaataagatgtgtgaaaatggacaaccatctaatacacggggaattaaggaaaaaaagaagaaagaagagcgtaagaagaagcgtactgttcaagaggaggaaagggaacgtggaaaagtgagctccaaagtttatttgtcatacatgggggaagcttacaaaggtacactgataccactaattatcttggctcaaaccatgttccaagttcttcagattgcgagcaactggtggatggcatgggcaaacccacaaacagaaggagatgctcccaagacagatagtgtggtccttctggttgtttatatgtcccttgcctttggaagttcactatttgtgttcatgagaagccttcttgtggctacgtttggtttagcagctgcccagaagctttttataaaaatgcttaggtgtgtctttcgagctccaatgtcattctttgacaccacaccatctggtcggattttgaacagagtttctgtagatcaaagtgttgtggaccttgatatagcgttcagacttggtggatttgcatcaacgacaattcaactccttggaattgttgctgtcatgagcaaagtcacatggcaagttctgattcttatagtccccatggctgttgcatgcatgtggatgcagaggtattatattgcttcatcaagggaactaactaggattttgagtgttcagaagtctccagtgatccatttgtttagtgaatcaattgctggtgctgctacaataaggggttttggtcaagagaagcggtttatgaaaaggaatctttatcttcttgactgttttgctcgccctttattttccagccttgctgctattgaatggctctgcctgcgaatggaattgctttcgactttcgtctttgctttttgcatggcaatacttgtgagctttcctcctggcacaatcgaaccaagtatggctggcctcgctgtaacatatggacttaatttaaatgctcgcatgtcaagatggatattgagcttctgtaaattagagaacaggataatctctgttgagcgcatttatcaatattgcaggcttcctagtgaagcaccattgattattgagaactgccgtccaccatcatcatggcctcagaatggaaacattgaactgattgatctcaaggtccgctacaaggacgatctaccattagttcttcatggtgtaagttgtatgtttcctggcgggaaaaagattgggattgtagggcgtactggaagcggtaaatctactcttattcaggcccttttccgcctaattgagcccactggagggaagattataattgacaacattgacatctctgcaattggccttcatgatctgcggtcacggttgagcatcattccccaagaccctacattgtttgagggtactatcagaatgaaccttgatcctcttgaggagtgcactgatcaagaaatttgggaggcactagaaaagtgtcagctaggagaggtcattcgttccaaggaagagaaacttgacagtccagtgctagaaaacggggataactggagcgtgggacagcgccaacttattgcactgggtagggcgctgctcaagcaggcaaaaattttggtactcgatgaggcgacagcatctgtcgacacagcaacagacaatcttatccaaaagatcatccgcagtgaattcaaggactgcacagtctgtaccattgctcaccgtattcccaccgttattgacagtgaccttgttctggtccttagtgatggtaaaatcgcagagttcgacacgccccagaggcttttagaggacaagtcatctatgttcatacagctagtatcggaatactccactcggtcgagctgtatatagagaggcttagcttaaaaccccgccccaaacctggcaacagaggctgggaggcaaatagcccgtatctgccatgcttgcgccatagaggtccctgcgaacaccggagggcggcgtagaagacgaggtgtacatgagtgggaggaacactgggcgttccctgacctgaataccgtggaatcggcgagggagcgcggttggtattggtaggcaccaggggaggagttggtgacactagtacattacccgaagctgatgcttcagtatgtatgtataacaacaatgcatactgcttctccctttgcagagtggagaaccaagggaataactcgtgcgtaataagaggagaaagatttgttttttggc

SEQ ID NO: 48 - DNA, 1313 bp, Zea mays
ctactactcaaatccatccttattgagcttagtgtttgatccatggactcggaaggagtagcagcaaaggtggcagatgagactactaaaccggcaagccaagaagacggcgagagcaaggccgggatgactgatctgctgatgctgaccgacaagtcgcagctgcaggcgctagcgatgctgctgcggaacaacgaggagctcatgatgagccaagcgatcaagtcggagacggagcgcgttgagtacctcaagacggtgagcgactgctacacgcggacaatgaagctccttgacgactccatggcggccaggatcacgtacgagcgttcgggcggaacgaggagcctcgtcgcccgggacatggacgactacgtcgtctacggcctcaacgcgtgcttgcagaacgtccgcaactgctgcgtgcgtctggacgccatcgacaagctgcgggcgcactacgacgccctcgccgacgccgtcgccgaaccggccgccaacgtcgagggcctcgccgcggaggcgtccgagtacaaggccgccatgtggcagtactgctacaaccagcggagcgcctccgcgcgggcgcactcccgcgcctactcccaggcgctcaagctggagggcatcgacttcgccgagcttgtgcggaggcaccagctccggctcgggtacggcagcaagggcgaggagttcgaggacctggacgacacccagaagctggaggtgtacaacagcatcatcgtcgagtcggggcgggcggggctaccggtgcggatgttctcgtcgggccgctctgccggtggccctaagattgcagccacgacgtgggcgcaggcggtgagcgtcttcatcatggcggcgggcaacctggcgtgggacgtgttcaccacggagcacgaggtggaggccatcctcaagggcagcctcaacctcctggcggggctagggggcttcgccgtggaggccgtcgtcggcgcggctgtcaccaaggcggtcgcaaacgtcggcgccggcgtctttgcttgctctctcgcgggcttcgtcgtgggcgccatagccgggctgatcttcatcggcgtcagcggcctcctcattaacctcatcatcggctcccctaggaaggtgcctgacatgagcaagctcatgttccacaccgccgtcatgcccgatggaatggcccttgcgtatgcggtatctcattaattacttattatcatcgcagtgactaccgatgcaactgcttcagatcctactgttggaacgcgtgtggaaataataaaggaataataataattattattgtaataaaa